SEQUENCE LISTING (13 sequences)

SEQ ID NO: 1 - protein, 116 aa, Unknown
  Description of Unknown ActRII polypeptide
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 2 - protein, 118 aa, Unknown
  Description of Unknown ActRII polypeptide
MTAPWVALALLWGSLWPSGRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 3 - protein, 122 aa, Unknown
  Description of Unknown ActRII polypeptide
MKFLVNVALVFMVVYISYIYASGRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 4 - protein, 123 aa, Unknown
  Description of Unknown ActRII polypeptide
MDAMKRGLCCVLLLCGAVFVSPSGRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 5 - protein, 225 aa, Unknown
  Description of Unknown Fc domain polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 6 - protein, 329 aa, Unknown
  Description of Unknown ActRII polypeptide
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 7 - protein, 344 aa, Unknown
  Description of Unknown ActRII polypeptide
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 8 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 9 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 10 - protein, 116 aa, Unknown
  Description of Unknown ActRII polypeptide
AILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 11 - protein, 135 aa, Unknown
  Description of Unknown ActRII polypeptide
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 12 - protein, 328 aa, Unknown
  Description of Unknown ActRII polypeptide
AILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMGGGTHTCPPCPAPELLGGPSVFLFPPKPKDTLMISRTPEVTCWVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 343 aa, Unknown
  Description of Unknown ActRII polypeptide
AILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPGGGTHTCPPCPAPELLGGPSVFLFPPKPKDTLMISRTPEVTCWVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2011/0268736 A1
(43) Pub. Date: Nov. 3, 2011

(54) METHOD FOR TREATING CONGENITAL MYOPATHY

(75) Inventors: Alan H. Beggs, Needham, MA (US); Michael W. Lawlor, Malden, MA (US)

(73) Assignee: CHILDREN'S MEDICAL CENTER CORPORATION, Boston, MA (US)

(21) Appl. No.: 13/097,322

(22) Filed: Apr. 29, 2011

Related U.S. Application Data
(60) Provisional application No. 61/330,011, filed on Apr. 30, 2010.

(51) Int. Cl. A61K 38/16; A61K 39/395; A61P 21/00; A61K 38/02

(52) U.S. Cl. 424/134.1; 514/1.1; 514/21.2

(57) ABSTRACT

The invention relates to methods and compositions for therapy for congenital myopathies.

### CROSS REFERENCE TO RELATED APPLICATIONS

This application claims benefit under 35 U.S.C. §119(e) of the U.S. Provisional Application No. 61/330,011, filed Apr. 30, 2010, the content of which is incorporated herein by reference in its entirety.

### GOVERNMENT SUPPORT

This invention was made with government support under grant no. P50 NS040828, no. R01 AR044345, no. L40 AR057721-01, and no. K08 AR059750-01 awarded by the National Institute of Arthritis and Musculoskeletal and Skin Diseases. The government has certain rights in this invention.

### FIELD OF THE INVENTION

The invention relates to methods for treating congenital myopathies.

### BACKGROUND OF THE INVENTION

X-linked myotubular myopathy (XLMTM) is a severe form of congenital myopathy with an estimated incidence of 1 in 50,000 male births, and most often manifests with severe perinatal weakness and respiratory failure. See, for example, J. Z. Heckmatt et al., Brain. 1985, 108(Pt 4):941-964 and H. Jungbluth, C. Wallgren-Pettersson & J. Laporte, Orphanet J Rare Dis. 2008, 3:26. Many patients with XLMTM die of the disease within the first year of life despite use of mechanical ventilation, and there are no US Food and Drug Administration-approved treatments for this disease. XLMTM is caused by mutations in the gene encoding myotubularin (MTM1), which is a phosphoinositide phosphatase thought to be involved in endosomal trafficking and/or maintenance of the sarcoplasmic reticulum and transverse tubular (T-tubular) system within myofibers. See, for example, A. Buj-Bello et al., Hum Mol Genet. 2008, 17:2132-2143; C. Cao et al., Mol Biol Cell. 2008, 19:3334-3346; J. J. Dowling et al., PLoS Genet. 2009, 5:e1000372; and J. Laporte et al., Nat Genet. 1996, 13:175-182. Muscle biopsy specimens from patients with XLMTM exhibit excessively small fibers with increased numbers of central nuclei and aggregation of organelles within the central regions of many cells (C. R. Pierson et al., J Neuropathol Exp Neurol. 2005, 64:555-564). While the number of centrally nucleated fibers bears little relationship to prognosis, there is a clear correlation between the degree of fiber smallness and severity of the disease (C. R. Pierson et al., Neuromuscul Disord. 2007, 17:562-568). A murine model of myotubularin deficiency, the Mtm1δ4 mouse (also referred to as Mtm1 KO) demonstrates features similar to those in human beings with the disease, including severe weakness, respiratory failure, and histologic findings that include myofiber smallness and increased numbers of centrally nucleated fibers. See, for example, A. Buj-Bello et al., Hum Mol Genet. 2008, 17:2132-2143; L. Al-Qusairi et al., Proc Natl Acad Sci USA. 2009, 106:18763-18768; and A. Buj-Bello et al., Proc Natl Acad Sci USA. 2002, 99:15060-15065. A new murine model of myotubularin deficiency, the Mtm1C205T mouse, has also been designed to model XLMTM patients with less severe disease.

Currently, there are no known cures for X-linked myotubular myopathy. Thus, there is a need for treatment of these disorders.

### SUMMARY OF THE INVENTION

In one aspect, the invention provides methods of treating a subject having a congenital myopathy, comprising administering to said subject an effective amount of an activin receptor type II (ActRII) inhibitor.

In some embodiments, the inhibitor binds an ActRII ligand.

In some embodiments, the inhibitor is a ActRII polypeptide or a portion thereof.

In some embodiments, the ActRII polypeptide or a portion thereof is selected from the group consisting of SEQ ID NO. 1-4, 6, and 7.

### BRIEF DESCRIPTION OF THE DRAWINGS

FIG. 1 is a line graph showing the body weight of vehicle and ActRIIB-mFc treated wild-type and Mtm1δ4 mice. Measurements shown are running averages±standard error of the mean (SEM) of (n) animals for each treatment group. (*p<0.05).

FIG. 2 is a line graph showing antigravity hanging performance of vehicle and ActRIIB-mFc treated wild-type and Mtm1δ4 mice. Measurements shown are mean values±SEM of (n) animals for each treatment group. (*p<0.05).

FIG. 3 is a line graph showing forelimb grip force of vehicle and ActRIIB-mFc treated wild-type and Mtm1δ4 mice. Measurements shown are mean values±SEM of (n) animals for each treatment group. (*p<0.05).

FIG. 4 is a line graph showing the number of foot drags seen per step in tested animals. Measurements shown are mean values±SEM of (n) animals for each treatment group. (*p<0.05).

FIG. 5 is a bar graph showing muscle weight of gastrocnemius, quadriceps and triceps in vehicle and ActRIIB-mFc treated wild-type and Mtm1δ4 mice. (*p<0.05).

FIGS. 6a-6b are frequency histograms of minferet diameter measurements from quadriceps muscles of vehicle and ActRIIB-mFc treated wild type (FIG. 5a) and Mtm1δ4 (FIG. 5b) mice at 43 days of life.

FIGS. 7a-7b are frequency histograms of minferet diameter measurements from quadriceps muscles of vehicle and ActRIIB-mFc treated wild type (FIG. 6a) and Mtm1δ4 (FIG. 6b) mice at end stage (˜9 weeks of life).

FIGS. 8a-8b are bar graphs showing number of myofibers in quadriceps of vehicle and ActRIIB-mFc treated wild-type and Mtm1δ4 mice at 43 days of life (FIG. 8a) and end stage (FIG. 8b).

FIG. 9 shows Kaplan-Meier survival curves for vehicle and ActRIIB-mFc treated and Mtm1δ4 mice. (*p<0.05).

FIG. 10 is a bar graph showing counts of centrally nucleated fibers in quadriceps muscle from mice at 43 days of life (DOL) and end stage. (*p<0.05).

FIG. 11 is a bar graph showing percentage of type 1, 2a, and 2b fibers.

FIGS. 12-14 are line graphs showing mean C-E: Muscles from animals at 35 days of life, 43 days of life, and end stage were analyzed. Mean MinFeret diameter measurements for type 1 (FIG. 12), type 2a (FIG. 13), and type 2b (FIG. 14) fibers from vehicle or ActRIIB-mFC treated WT or Mtm1δ4 animals at 35 days of life, 43 days of life, and end stage. (*p<0.01; **p<0.0001).

FIG. 15 is a line graph showing animal mass in RAP-031 treated Mtm1C205T mice. Measurements of animal mass in vehicle (VEH) or RAP-031 (ActRIIB-mFC)-treated wild type (WT) or Mtm1C205T mice show that RAP-031 treated WT and Mtm1C205T mice had higher average weights than their vehicle treated counterparts. (*P<0.05)

FIG. 16 is a bar graph showing weight of gastrocnemius muscle following treatment of Mtm1C205T Mice with ActRIIB-mFC (RAP-031). Muscle masses taken at 6 months of life reveal that ActRIIB-mFC treatment of Mtm1C205T mice produced an increase in mass in the gastrocnemius muscle. (*p<0.05)

### DETAILED DESCRIPTION OF THE INVENTION

In one aspect, the invention provides a method of treating a subject having a congenital myopathy, comprising administering to said subject an effective amount of an activin receptor type II (ActRII) inhibitor.

In some embodiments, the method further comprises the step of selecting a subject that has been diagnosed with a congenital myopathy.

In some embodiments, the method also comprises the step of diagnosing a subject for congenital myopathy before onset of administering the ActRII inhibitor.

In some embodiments, the subject is already being treated with an ActII inhibitor. However, it is necessary that the subject is not being treated for a congenital myopathy. In some other embodiments, the selected subject is not being treated with a ActII inhibitor.

Studies in MTM1 knockout mice have suggested that a component of the pathogenesis of some instances of congenital myopathy may be due to an inability to attain or maintain proper myofiber size (Buj-Bello, A., et al., The lipid phosphatase myotublarin is essential for skeletal muscle maintenance but not for myogenesis in mice. Proc. Natl. Acad. Sci. U.S.A. 2003, vol 99, pp 15060-15065). Patients with larger myofibers tend to have better outcome. Thus, without wishing to be bound by theory, promoting muscle hypertrophy can inhibit or attenuate symptoms of congenital myopathy. In some embodiments, the inhibitor promotes muscle hypertrophy. Muscle hypertrophy can be determined from an increase in muscle mass or weight in the treated subject relative to untreated subject. The increase can be in all muscles or in some muscles only. In some embodiments, the inhibitor increases mass of muscle selected from the group consisting of quadriceps, gastrocnemius, triceps, soleus, extensor digitorum longus, tibialis anterior, diaphragm muscles, and combinations there of. In some embodiments, inhibitor increases the muscle mass or weight by at least 10%, 20%, 30%, 40%, 50%, 100%, 2-fold, 3-fold, 5-fold or more. Increase in muscle mass can also lead to increase in weight of treated subjected relative to untreated subject.

Studies have also shown that myofiber size correlates with MTM1 mutation type and outcome in XLMTM (Pierson, C. R., et al., Neuromuscul. Disord., 2007, vol 17, pp 562-568.) Accordingly, one effect of treatment by administration of inhibitor is an increase in the size of type I and/or type II myofibers. By an increase in size of myofiber is meant that average size of the myofiber in the treated subject is higher than the average size of the myofiber in the untreated subject. The increase in myofibers can occur with or without an increase in number of myofibers.

Accordingly, in some embodiments, the myofibers increase in size by at least 10%, at least 20%, at least 30%, at least 40%, at least 50%, at least 60%, at least 70%, at least 80%, at least 90%, at least 1-fold, at least 2-fold, at least 3-fold, at least 4-fold, or at least 5-fold or more on administration of an ActRII inhibitor relative to an untreated control or relative to before onset of ActRII inhibitor administration.

In some embodiments, the number of myofibers is increased by at least 10%, at least 20%, at least 30%, at least 40%, at least 50%, at least 60%, at least 70%, at least 80%, at least 90%, at least 1-fold, at least 2-fold, at least 3-fold, at least 4-fold, or at least 5-fold or more on administration of an ActRII inhibitor relative to the untreated control or relative to before onset of ActRII inhibitor administration.

While all types of myofibers may increase in size and/or number on administration of an ActRII inhibitor, the inventors have discovered that ActRII inhibitors preferentially increase the size and/or number of type 2b myofibers in an animal model of X-linked myotubular myopathy. According, in some embodiments, type 2b myofibers increase in size by at least 10%, at least 20%, at least 30%, at least 40%, at least 50%, at least 60%, at least 70%, at least 80%, at least 90%, at least 1-fold, at least 2-fold, at least 3-fold, at least 4-fold, or at least 5-fold or more on administration of an ActRII inhibitor relative to an untreated control or relative to before onset of ActRII inhibitor administration.

In some embodiments, the number of type 2b myofibers is increased by at least 10%, at least 20%, at least 30%, at least 40%, at least 50%, at least 60%, at least 70%, at least 80%, at least 90%, at least 1-fold, at least 2-fold, at least 3-fold, at least 4-fold, or at least 5-fold or more on administration of an ActRII inhibitor relative to the untreated control or relative to before onset of ActRII inhibitor administration.

In some embodiments, type 1 and/or type 2a myofibers do not increase in size on administration of an ActRII inhibitor relative to an untreated control or relative to before onset of ActRII inhibitor administration. By not increasing in size is meant that the mean MinFeret diameter of type1 and/or type 2b myofibers is within 10% of the mean MinFeret diameter of such myofibers relative to an untreated control or relative to before onset of ActRII inhibitor administration.

In some embodiments, the number of type 1 and/or type 2a myofibers does not increase on administration of an ActRII inhibitor relative to the untreated control or relative to before onset of ActRII inhibitor administration. By not increasing in number is meant that number of type 1 and/or type 2b myofibers is within 10% of the number of such myofibers relative to an untreated control or relative to before onset of ActRII inhibitor administration.

In some embodiments, type 2b myofibers increase in size by at least 10%, at least 20%, at least 30%, at least 40%, at least 50%, at least 60%, at least 70%, at least 80%, at least 90%, at least 1-fold, at least 2-fold, at least 3-fold, at least 4-fold, or at least 5-fold or more on administration of an ActRII inhibitor relative to increase in size of type 1 and/or type 2a myofibers on administration of an ActRII inhibitor.

In some embodiments, number of type 2b myofibers increases by at least 10%, at least 20%, at least 30%, at least 40%, at least 50%, at least 60%, at least 70%, at least 80%, at least 90%, at least 1-fold, at least 2-fold, at least 3-fold, at least 4-fold, or at least 5-fold or more on administration of an ActRII inhibitor relative to increase in number of type 1 and/or type 2a myofibers on administration of an ActRII inhibitor.

Without wishing to be bound by theory, benefits of treatment can include gain in weight, grip strength, or prolongation of lifespan.

In some embodiments, congenital myopathy is selected from the group consisting of myotubular myopathy, centronuclear myopathy, central core myopathy, nemaline myopathy, multiminicore myopathy (e.g. minicore myopathy, multicore myopathy, multimincore disease, sepn1-related myopathy), congenital fiber type disproportion, and any combinations thereof.

### Myostatin

Myostatin (formerly termed “growth differentiation factor 8”) is a protein of the transforming growth factor beta (TGF-β) superfamily that is selectively expressed in skeletal muscle, cardiac muscle, and adipose tissue during late embryogenesis and adulthood and is an important negative regulator of myofiber size. See for example, A. C. McPherron, A. M. Lawler & S. J. Lee, Nature. 1997, 387:83-90. Myostatin binds to and signals through the activin type IIB receptor (ActRIIB) to activate the TGF-β pathway, which prevents progression through the cell cycle and down-regulates several key processes related to myofiber hypertrophy. See S. McCroskery et al., J Cell Biol. 2003, 162:1135-1147 and D. Joulia-Ekaza & G. Cabello, Exp Cell Res. 2006; 312:2401-2414. Examples of myostatin deficiency found in sheep (A. Clop et al., Nat Genet. 2006, 38:813-818), mice (A. C. McPherron, A. M. Lawler & S. J. Lee, Nature. 1997, 387:83-90), cattle, dogs, and one human child have all exhibited generalized muscular hypertrophy and increased strength (D. Joulia-Ekaza & G. Cabello G, Exp Cell Res. 2006, 312:2401-2414), and compared with mice with intact myostatin expression, null mice for myostatin or ActRIIB demonstrate myofiber hypertrophy and hyperplasia (S. J. Lee & A. C. McPherron, Proc Natl Acad Sci USA. 2001, 98:9306-9311 and S. J. Lee et al., Proc Natl Acad Sci USA. 2005, 102:18117-18122). There seems to be myostatin-independent suppression of muscle growth that is mediated by ActRIIB Treatment with a soluble ActRIIB-mFC increases muscle mass in myostatin knockout mice (S. J. Lee et al., Proc Natl Acad Sci USA. 2005, 102:18117-18122), which supports the role of multiple ligands that control muscle growth postnatally and suggests that targeting ActRIIB rather than myostatin alone may provide additional therapeutic benefit. The potential for myostatin inhibition to promote muscle growth has led to development of a new class of myostatin and ActRIIB inhibitors as prospective therapeutic agents for myopathic, dystrophic, and neurologic disorders. A soluble activin-receptor type IIB fusion protein (ActRIIB-mFC) has been developed that potently binds to TGF-β family members to produce muscle fiber growth in vitro and in vivo. See S. M. Cadena et al., J Appl Physiol. 2010, 109:635-642. Recent studies using ActRIIB-mFC in murine models of neurologic disorders including amyotrophic lateral sclerosis and spinal muscular atrophy have demonstrated variable effects on muscle strength and no effects on animal survival. See, for example, B. M. Morrison et al., Exp Neurol. 2009, 217:258-268 and C. J. Sumner et al., Hum Mol Genet. 2009, 18:3145-3152. The effectiveness of this agent for treatment of primary disorders of skeletal muscle is also being investigated.

Myostatin is a natural protein that functions to negatively regulate the size of skeletal muscle by binding to a receptor on the cell surface, triggering a series of secondary events leading to gene activation and inhibition in a pattern that stops muscle growth. Several animal models, and one known human patient, with mutations in the myostatin gene are characterized by markedly increased muscle mass and strength. Numerous studies have shown that inhibiting myostain through various means results in increased muscle growth, mass and strength. Recent studies have elucidated an important role for ActRIIB in muscle growth regulation. Thus, without wishing to be bound by theory, promoting muscle hypertrophy can attenuate symptoms resulting from myotubularin deficiency. As the inventors have discovered, one way to promote hypertrophy is by inhibiting the ActRIIB

### Congenital Myopathy

Congenital myopathy is a term sometimes applied to hundreds of distinct neuromuscular disorders that may be present at birth, but it is usually reserved for a group of rare inherited primary muscle disorders that cause hypotonia and weakness at birth or during the neonatal period and, in some cases, delayed motor development later in childhood. Patients suffer from weakness ranging from mild (late childhood onset and ability to walk through adulthood) to severe (respitatory insufficiency and death within the first year of life).

The most common types of congenital myopathy are nemaline myopathy, myotubular myopathy, central core myopathy, congenital fiber type disproportion, and multicore myopathy. They are distinguished primarily by their histological features, symptoms, and prognosis. Diagnosis is indicated by characteristic clinical findings and confirmed by muscle biopsy.

X-Linked Myotubular myopathy (XLMTM): Myotubular myopathy is autosomal or X-linked. The more common autosomal variation produces mild weakness and hypotonia in both sexes. The X-linked variation affects males and results in severe skeletal muscle weakness and hypotonia, facial weakness, impaired swallowing, and respiratory muscle weakness and respiratory failure. However, female carriers rarely express significant clinical symptoms. Most patients die within the first year of life from respiratory failure. Some patients survive for several years and may show spontaneous improvement of respiratory function after birth. XLMTM has also been referred to as CNM, MTMX, X-linked centronuclear myopathy, and XMTM in the art.

The characteristic muscle histopathology consists of small rounded muscle fibers with centrally located nuclei surrounded by a halo devoid of contractile elements but containing mitochondria. The MTM1 gene is mutated in vast majority of XLMTM patients. This gene is ubiquitously expressed and shows a muscle-specific alternative transcript due to the use of a different polyadenylation signal. Over one hundred thirty-three different disease associated mutations have been described in the MTM1 gene. A list of MTM1 mutations is maintained on the web at the Human Gene Mutation Database under entry for XLMTM and can be accessed at the following address: www.uwcm.ac.uk/uwcm/mg/search/119439.html. Mutations in the MTM1 gene are widespread throughout the gene, although more have been found in exon 4, 12, 3, 8, 9, and 11, in that order when comparing the number of mutations to the nucleotide-length ratio for each exon. For a review of MTM1 mutations in X-Linked Myotubular Myopathy see J. Laporte et al. 2000, 15:393-409.

Mutations in the MTM1 gene include missense, nonsense, small insertions or deletions, large deletions and splice-site mutations. Most point mutations are truncating; however about 25% of the mutations are missense. While most truncating and splice mutations are associated with a severe phenotype, some missense mutations are associated with milder or less severe phenotype and prolonged survival.

Nemaline myopathy: Nemaline myopathy can be autosomal dominant or recessive and result from various mutations on different chromosomes. Nemaline myopathy may be severe, moderate, or mild in neonates. Severely affected patients may experience weakness of respiratory muscles and respiratory failure. Moderate disease produces progressive weakness in muscles of the face, neck, trunk, and feet, but life expectancy may be nearly normal. Mild disease is nonprogressive, and life expectancy is normal.

Central core myopathy: Inheritance is autosomal dominant. Most affected patients develop hypotonia and mild proximal muscle weakness as neonates. Many also have facial weakness. Weakness is nonprogressive, and life expectancy is normal. However, patients are at higher risk of developing malignant hyperthermia (the gene associated with central core myopathy is also associated with increased susceptibility to malignant hyperthermia).

Congenital fiber type disproportion: Congenital fiber type disproportion is inherited, but the pattern is poorly understood. Hypotonia and weakness of the face, neck, trunk, and limbs are often accompanied by skeletal abnormalities and dysmorphic features. Most affected children improve with age, but a small percentage develops respiratory failure.

Multicore myopathy: Multicore myopathy is usually autosomal recessive but may be autosomal dominant. Infants typically present with proximal weakness, but some children present later with generalized weakness. Progression is highly variable.

### Symptoms of Congenital Myopathy

In some embodiments, a method described herein also comprises the step of diagnosing a subject for congenital myopathy before onset of administering the ActRII inhibitor. A subject can be diagnosed for congenital myopathy based on the symptoms presented by the subject.

Generally, symptoms of congenital myopathies include, but are not limited to, depressed reflexes, enlarged muscles, difficulty relaxing muscles following contractions, stiff muscles, and rigid muscles. Specific symptoms are described below.

Central core disease is characterized by a mild, non-progressive muscle weakness. Signs of central core disease usually appear in infancy or early childhood and may present even earlier. There may be decreased fetal movements and breech (feet first) presentation in utero. The main features of CCD are poor muscle tone (hypotonia), muscle weakness, and skeletal problems including congenital hip dislocation, scoliosis (curvature of the spine), pes cavus (high-arched feet), and clubbed feet. Children with CCD experience delays in reaching motor milestones and tend to sit and walk much later than those without the disorder. A child with the disease usually cannot run easily, and may find that jumping and other physical activities are often impossible. Although central core disease may be disabling, it usually does not affect intelligence or life expectancy.

People who have central core disease are sometimes vulnerable to malignant hyperthermia (MH), a condition triggered by anesthesia during surgery. MH causes a rapid, and sometimes fatal, rise in body temperature, producing muscle stiffness.

There is variability in age of onset, presence of symptoms, and severity of symptoms in nemaline myopathy (NM). Most commonly, NM presents in infancy or early childhood with weakness and poor muscle tone. In some cases there may have been pregnancy complications such as polyhydramnios (excess amniotic fluid) and decreased fetal movements. Affected children with NM tend to have delays in motor milestones such as rolling over, sitting and walking. Muscle weakness commonly occurs in the face, neck and upper limbs. Over time, a characteristic myopathic face (a long face that lacks expression) develops. Skeletal problems including chest deformities, scoliosis, and foot deformities may develop. In the most severe cases of NM, feeding difficulties and potentially fatal respiratory problems may also occur. In those who survive the first two years of life, muscle weakness tends to progress slowly or not at all.

Typically the X-linked form of MTM (XLMTM) is the most severe of the three forms (X-linked, autosomal recessive, and autosomal dominant). XLMTM usually presents as a newborn male with poor muscle tone and respiratory distress. The pregnancy may have been complicated by polyhydramnios and decreased fetal movements. Of those who survive the newborn period, many will at least partially depend on a ventilator for breathing. Because of the risk of aspiration, many will also have a gastrostomy tube (G-tube). Boys with XLMTM can experience significant delays in achieving motor milestones and may not ever walk independently. They tend to be tall with a characteristic facial appearance (long, narrow face with a highly arched roof of the mouth and crowded teeth). Intelligence is generally not affected. Medical complications that may develop include: scoliosis, eye problems (eye muscle paralysis and droopy eyelids), and dental malocclusion (severe crowding). In XLMTM, other problems including undescended testicles, spherocytosis, peliosis, elevated liver enzymes, and gallstones may occur.

The autosomal recessive and autosomal dominant forms of MTM tend to have a milder course than the X-linked form. The autosomal recessive form can present in infancy, childhood, or early adulthood. Common features include generalized muscle weakness with or without facial weakness and ophthalmoplegia (paralysis of the eye muscles). Although feeding and breathing problems can occur, affected individuals usually survive infancy. Onset of the autosomal dominant form ranges from late childhood through early adulthood. It tends to be the mildest of the three forms of MTM. Unlike the X-linked form of the condition, problems with other organs (such as the liver, kidneys, and gall bladder) haven't been reported with the autosomal recessive and autosomal dominant forms of MTM.

### Diagnosis of Congenital Myopathy

Diagnosis of a congenital myopathy generally includes evaluation of the subject's personal and family history, physical and neurological examinations that test reflexes and strength, and specialized tests. Since there is overlap between the symptoms of a congenital myopathy and other neuromuscular disorders, a number of tests may be performed to help narrow down the diagnosis. Serum CK (creatinine kinase) analysis, EMG (electromyelogram), nerve conduction studies, and muscle ultrasound tend to be of limited value in making this diagnosis. The definitive diagnosis of a congenital myopathy usually relies upon genetic testing and/or muscle biopsy. Also, muscle biopsy can be used to determine a patient's susceptibility to malignant hyperthermia.

X-linked myotubular myopathy: Diagnosis of X-linked MTM is usually made on muscle biopsy. Findings include: centrally located nuclei in muscle fibers that look like myotubules, absence of structures known as myofibrils, and possibly, persistence of certain proteins usually seen in fetal muscle cells. Gene testing detects a mutation (disease-causing gene change) in up to 97-98% of people with the X-linked form. Gene testing can comprise: (i) complete gene sequencing of the MTM1 gene; (ii) mutation screening (scanning) by methods such as single-stranded conformational polymorphism (SSCP) or denaturing gradient high-performance liquid chromatography (DHPLC), followed by sequencing of the abnormal fragments; and (iii) deletion testing. XLMTM can also be diagnosed by measuring levels of myoubilarin. Patients with known MTM1 mutations usually show abnormal myotubularin levels.

Central core disease: The muscle biopsy from a person with CCD typically displays a metabolically inactive “core” or central region that appears blank when stained (tested) for certain metabolic enzymes (proteins) that should be there. These central regions also lack mitochondria, the energy producing “factories” of the cells. Genetic testing for RYR1 mutations is available on a research basis. The same genetic test may be used to determine the presence of the gene change in family members who may have or be at-risk for the disease. For families in which a RYR1 mutation has been found, prenatal diagnosis may be possible using the DNA of fetal cells obtained from chorionic villus sampling (CVS) or amniocentesis.

Nemaline myopathy: The clinical diagnosis of NM is suspected in an infant under age one with muscle weakness and hypotonia (decreased muscle tone). Definitive diagnosis of nemaline myopathy is made by demonstration of nemaline bodies, rod-shaped structures characteristic of this disease, using a specific stain known as “Gomori trichrome” on a muscle biopsy sample. Muscle biopsy may also show predominance of structures known as type I fibers. Genetic testing is available on a clinical basis for one gene, the ACTA1 gene located on the long arm of chromosome 1. About 15% of NM cases are due to mutations in this gene. Prenatal diagnosis is possible for families with known ACTA1 mutations. The DNA of a fetus can be tested using cells obtained from chorionic villus sampling (CVS) or amniocentesis.

ActII Inhibitors

As used herein, the term “ActRII” refers to a family of activin receptor type II (ActRII) proteins and ActRII-related proteins derived from any species. Reference to ActRII herein is understood to be reference to any one of the currently identified forms, including ActRIIA and ActRIIB. Members of the ActRII family are generally all transmembrane proteins composed of a ligand-binding extracellular domain with cysteine-rich region, a transmembrane domain, and a cytoplasmic domain with predicted serine/kinase specificity. Amino acid sequences of human ActRIIA precursor protein and ActRIIB precursor protein are shown as SEQ ID NO: 8 (NP_{—}001607, 513aa) and SEQ ID NO: 9 (NP_{—}001097, 512aa), respectively.

In some preferred embodiments, ActRII is ActRIIB.

By “ActRII inhibitor” is meant any compound that inhibits (or antagonizes) an ActRII receptor in any process associated with ActRII activity. For example, inhibition of ActRII receptors inhibits ActRII signaling, such as intracellular signal transduction events triggered by an ActRII ligand. Preferably, ActRII signaling is lowered or inhibited by at least about 5%, 10%, 20%, 30%, 40%, 50%, 60%, 70%, 80%, 90% or higher, relative to when ActRII is not inhibited.

The inhibitor can act in a number of different ways to antagonize ActRII receptors. For examples, the inhibitor can inhibit the receptor by binding to the receptor at the ligand binding site, by binding receptor and altering the ligand binding site, by binding to the ligand, by inhibiting the expression of a gene encoding ActRII receptor, and/or by inhibiting the expression of a gene encoding a ActRII receptor ligand. Exemplary ligands of ActRII receptors include some TGF-beta family members, such as activin, Nodal, GDF8 (also known as myostatin), GDF11, and BMP7. In one embodiment, the inhibitor binds GDF-8.

In some embodiments, the inhibitor binds an ActRII ligand. An inhibitor can bind to an ActRII receptor ligand with a Kd less than 100 μM, less than 10 μM, less than 1 μM, less than 100 nM, less than 10 nM, less than 1 nM, less than 100 pM, less than 10 pM, or less than 1 pM. Without wishing to be bound by theory, the inhibitor binds to a ActRII receptor ligand and antagonizes an ActRII receptor in any process associated with ActRII activity.

The ActRII inhibitor can be a small organic or inorganic molecule, a peptide, a polypeptide, a protein, peptide analog and/or derivative thereof, peptidomimetic, nucleic acid, nucleic acid analog and/or derivative, polynucleotide, oligonucleotide, or any combinations thereof. The ActRII inhibitor can be hydrophobic, hydrophilic, or amphiphilic.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is highly preferred that a small molecule have a molecular mass equal to or less than 700 Daltons.

As used herein, the term “peptide” is used in its broadest sense to refer to compounds containing two or more amino acids, amino acid equivalents or other non-amino groups joined to each other by peptide bonds or modified peptide bonds. Peptide equivalents can differ from conventional peptides by the replacement of one or more amino acids with related organic acids (such as PABA), amino acids or the like or the substitution or modification of side chains or functional groups. A peptide can be of any size so long; however, in some embodiments, peptides having twenty or fewer total amino acids are preferred. Additionally, the peptide can be linear or cyclic. Peptide sequences specifically recited herein are written with the amino terminus on the left and the carboxy terminus on the right.

In addition, the term “peptide” broadly includes proteins, which generally are polypeptides. As used herein, the term “protein” is used to describe proteins as well as fragments thereof. Thus, any chain of amino acids that exhibits a three dimensional structure is included in the term “protein”, and protein fragments are accordingly embraced.

A peptidomimetic is a molecule capable of folding into a defined three-dimensional structure similar to a natural peptide As used herein, the term “nucleic acid” refers to a polymers (polynucleotides) or oligomers (oligonucleotides) of nucleotide or nucleoside monomers consisting of naturally occurring bases, sugars and intersugar linkages. The term “nucleic acid” also includes polymers or oligomers comprising non-naturally occurring monomers, or portions thereof, which function similarly. Such modified or substituted nucleic acids are often preferred over native forms because of properties such as, for example, enhanced cellular uptake and increased stability in the presence of nucleases.

A nucleic acid can be single-stranded or double-stranded. A single-stranded nucleic acid can have double-stranded regions and a double-stranded nucleic acid can have single-stranded regions. Exemplary nucleic acids include, but are not limited to structural genes, genes including control and termination regions, self-replicating systems such as viral or plasmid DNA, single-stranded and double-stranded siRNAs and other RNA interference reagents (RNAi agents or iRNA agents), short-hairpin RNAs (shRNA), antisense oligonucleotides, ribozymes, microRNAs, microRNA mimics, aptamers, antimirs, antagomirs, triplex-forming oligonucleotides, RNA activators, immuno-stimulatory oligonucleotides, and decoy oligonucleotides.

In some embodiments, the inhibitor is an ActRII polypeptide or a portion or fragment thereof. As used herein, the term “ActRII polypeptide” refers to polypeptides comprising any naturally occurring polypeptide of an ActRII family member as well as any variants thereof (including mutants, fragments, fusions, peptidomimetic forms, and combinations thereof) that retain a useful activity. For example, ActRII polypeptides include polypeptides derived from the sequence of any known ActRII having a sequence at least about 75%, 80%, 85%, 90%, 95%, or 99% identical to the sequence of the known ActRII. Examples of ActRII polypeptides include the naturally occurring ActRII polypeptides as well as functional variants thereof. ActRII polypeptides also include polypeptides that comprise at least 10, at least 15, at least 20, at least 25, at least 30, at least 35, at least 40, at least 45, at least 50, or at least 60 consecutive amino acids of a sequence selected from SEQ ID Nos. 1-4, 6-13.

TABLE 1
Polypeptide sequences.

| SEQ | |
|---|---|
| ID | |
| No. | Sequence |
| 1 | SGRGEAETRECIYYNANWELERTNQSGLERCEGEQDKRLHCYASWANSSGTIELVKK |
| | GCWLDDFNCYDRQECVATEENPQVYFCCCEGNFCNERFTHLPEAGGPEVTYEPPPTA |
| | PT |
| 2 | MTAPWVALALLWGSLWPSGRGEAETRECIYYNANWELERTNQSGLERCEGEQDKRL |
| | HCYASWANSSGTIELVKKGCWLDDFNCYDRQECVATEENPQVYFCCCEGNFCNERFT |
| | HLPEA |
| 3 | MKFLVNVALVFMVVYISYIYASGRGEAETRECIYYNANWELERTNQSGLERCEGEQ |
| | KRLHCYASWANSSGTIELVKKGCWLDDFNCYDRQECVATEENPQVYFCCCEGNFCN |
| | ERFTHLPEA |
| 4 | MDAMKRGLCCVLLLCGAVFVSPSGRGEAETRECIYYNANWELERTNQSGLERCEGEQ |
| | DKRLHCYASWANSSGTIELVKKGCWLDDFNCYDRQECVATEENPQVYFCCCEGNFC |
| | NERFTHLPEA |
| 5 | THTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPVPIEKTIS |
| | KAKGQPREPQVYTEPPSREEMTKNQVSLTCLVKGFYPSDIAVEWESNGQPENNYKTTP |
| | PVLDSDGPFFLYSKLTVDKSRWQQGNVFSCSVMHEALHNHYTQKSLSLSPGK |
| 6 | SGRGEAETRECIYYNANWELERTNQSGLERCEGEQDKRLHCYASWANSSGTIELVKK |
| | GCWLDDFNCYDRQECVATEENPQVYFCCCEGNFCNERFTHLPEAGGGTHTCPPCPAP |
| | ELLGGPSVFLFPPKPKDTLMISRTPEVTCVVVDVSHEDPEVKFNWYVDGVEVHNAKT |
| | KPREEQYNSTYRVVSVETVLHQDWENGKEYKCKVSNKALPVPIEKTISKAKGQPREP |
| | QVYTEPPSREEMTKNQVSLTCLVKGFYPSDIAVEWESNGQPENNYKTTPPVLDSDGPF |
| | FLYSKLTVDKSRWQQGNVFSCSVMHEALHNHYTQKSLSLSPGK |
| 7 | SGRGEAETRECIYYNANWELERTNQSGLERCEGEQDKRLHCYASWANSSGTIELVKK |
| | GCWLDDFNCYDRQECVATEENPQVYFCCCEGNFCNERFTHLPEAGGPEVTYEPPPTA |
| | PTGGGTHTCPPCPAPELLGGPSVFLFPPKPKDTLMISRTPEVTCVVVDVSHEDPEVKFN |
| | WYVDGVEVHNAKTKPREEQYNSTYRVVSVETVLHQDWENGKEYKCKVSNKALPVPI |
| | EKTISKAKGQPREPQVYTEPPSREEMTKNQVSLTCLVKGFYPSDIAVEWESNGQPENN |
| | YKTTPPVLDSDGPEFLYSKETVDKSRWQQGNVFSCSVMHEALHNHYTQKSLSESPGK |
| 8 | MGAAAKLAFAVFLISCSSGAILGRSETQECLFFNANWEKDRTNQTGVEPCYGDKDKR |
| | RHCFATWKNISGSIEIVKQGCWLDDINCYDRTDCVEKKDSPEVYFCCCEGNMCNEKFS |
| | YFPEMEVTQPTSNPVTPKPPYYNILLYSLVPLMLIAGIVICAFWVYRHHKMAYPPVLVP |
| | TQDPGPPPPSPLEGLKPLQLLEVKARGRFGCVWKAQLLNEYVAVKIFPIQDKQSWQNE |
| | YEVYSLPGMKHENILQFIGAEKRGTSVDVDLWLITAFHEKGSLSDFLKANVVSWNELC |
| | HIAETMARGLAYLHEDIPGLKDGHKPAISHRDIKSKNVELKNNETACIADFGLALKFE |
| | AGKSAGDTHGQVGTRRYMAPEVLEGAINFQRDAFERIDMYAMGLVLWELASRCTAA |
| | DGPVDEYMLPFLEEIGQHPSLEDMQEVVVHKKKRPVERDYWQKHAGMAMECETIEE |
| | CWDHDAEARLSAGCVGERITQMQRLTNIITTEDIVTVVTMVTNVDFPPKESSL |
| 9 | MTAPWVALALLWGSLCAGSGRGEAETRECIYYNANWELERTNQSGLERCEGEQDKR |
| | LHCYASWRNSSGTIELVKKGCWLDDFNCYDRQECVATEENPQVYFCCCEGNFCNERF |
| | THLPEAGGPEVTYEPPPTAPTLLTVLAYSLLPIGGLSLIVLLAFWMYRHRKPPYGHVDI |
| | HEDPGPPPPSPLVGLKPLQLLEIKARGRFGCVWKAQLMNDFVAVKIFPLQDKQSWQSE |
| | REIFSTPGMKHENLLQFIAAEKRGSNLEVELWLITAFHDKGSLTDYLKGNIITWNELCH |
| | VAETMSRGLSYLHEDVPWCRGEGHKPSIAHRDFKSKNVLLKSDLTAVLADFGLAVRF |
| | EPGKPPGDTHGQVGTRRYMAPEVLEGAINFQRDAFERIDMYAMGLVLWELVSRCKA |
| | ADGPVDEYMLPFLEEIGQHPSLEELQEVVVHKKMRPTIKDHWLKHPGLAQLCVTIEEC |
| | WDHDAEARLSAGCVEERVSLIRRSVNGTTSDCLVSLVTSVTNVDLPPKESSI |
| 10 | AILGRSETQECLFFNANWEKDRTNQTGVEPCYGDKDKRRHCFATWKNISGSIEIVKQG |
| | CWLDDINCYDRTDCVEKKDSPEVYFCCCEGNMCNEKFSYFPEMEVTQPTSNPVTPKPP |
| 11 | MGAAAKLAFAVFLISCSSGAILGRSETQECLFFNANWEKDRTNQTGVEPCYGDKDKR |
| | RHCFATWKNISGSIEIVKQGCWLDDINCYDRTDCVEKKDSPEVYFCCCEGNMCNEKFS |
| | YFPEMEVTQPTSNPVTPKPP |
| 12 | AILGRSETQECLFFNANWEKDRTNQTGVEPCYGDKDKRRHCFATWKNISGSIEIVKQG |
| | CWLDDINCYDRTDCVEKKDSPEVYFCCCEGNMCNEKFSYFPEMGGGTHTCPPCPAPE |
| | LLGGPSVFLFPPKPKDTLMISRTPEVTCWVDVSHEDPEVKFNWYVDGVEVHNAKTKP |
| | REEQYNSTYRVVSVLTVLHQDWLNGKEYKCKVSNKALPVPIEKTISKAKGQPREPQV |
| | YTEPPSREEMTKNQVSLTCLVKGFYPSDIAVEWESNGQPENNYKTTPPVLDSDGSEFL |
| | YSKLTVDKSRWQQGNVFSCSVMHEALHNHYTQKSLSLSPGK |
| 13 | AILGRSETQECLFFNANWEKDRTNQTGVEPCYGDKDKRRHCFATWKNISGSIEIVKQG |
| | CWLDDINCYDRTDCVEKKDSPEVYFCCCEGNMCNEKFSYFPEMEVTQPTSNPVTPKP |
| | PGGGTHTCPPCPAPELLGGPSVFLFPPKPKDTLMISRTPEVTCWVDVSHEDPEVKFNW |
| | YVDGVEVHNAKTKPREEQYNSTYRVVSVLTVLHQDWLNGKEYKCKVSNKALPVPIE |
| | KTISKAKGQPREPQVYTLPPSREEMTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |

Additional ActRII polypeptides that are amenable to the invention are described in U.S. Patent Publication No. 2006/0068468; No. 2008/0089897; and No. 2009/0005308, content of all of which is incorporated herein by reference in their entirety.

In some embodiments, a functional variant of ActRII polypeptide has an amino acid sequence that is at least 75% identical to an amino acid sequence selected from SEQ ID Nos. 1-4 and 6-13. In some cases, the functional variant has an amino acid sequence at least 80%, 85%, 90%, 95%, 99% or 100% identical to an amino acid sequence selected from the group consisting of SEQ ID NO: 1-4 and 6-13.

ActRII polypeptide can be a soluble ActRII polypeptide. As used herein, the term “soluble ActRII polypeptide” refers to polypeptides comprising an extracellular domain of an ActRII protein. Soluble ActRII polypeptides include any naturally occurring extracellular domain of an ActRII protein as well as any variants thereof (including mutants, fragments, fusions, peptidomimetic forms, and combinations thereof) that retain a useful activity. For example, the extracellular domain of an ActRII protein binds to a ligand and is generally soluble. Examples of soluble ActRII polypeptides include ActRIIA and ActRIIB soluble polypeptides illustrated as SEQ ID NO: 10 and SEQ ID NO: 1, respectively.

In some embodiments, the soluble ActRII polypeptide can comprise a signal sequence in addition to the extracellular domain of an ActRII protein. The signal sequence can be a native signal sequence of an ActRII, or a signal sequence from another protein such as tissue plasminogen activator (TPA) signal sequence or a honey bee melatin (HBM) signal sequence. Examples of soluble ActRII polypeptides that comprise a signal sequence in addition to the extracellular domain of an ActRII protein include the sequences illustrated for ActRIIB as SEQ ID NO: 2, SEQ ID NO: 3, and SEQ ID NO: 4, and for ActRIIA as SEQ ID NO: 11.

In some embodiments, ActRII polypeptide is a fusion protein. An ActRII fusion protein has, as one domain, an ActRII polypeptide (e.g., a ligand-binding portion of an ActRII) and one or more additional domains that provide a desirable property, such as improved pharmacokinetics, easier purification, targeting to particular tissues, etc. For example, a domain of a fusion protein may enhance one or more of in vivo stability, in vivo half life, uptake/administration, tissue localization or distribution, formation of protein complexes, multimerization of the fusion protein, and/or purification. An ActRII fusion protein can include an immunoglobulin Fc domain (wild-type or mutant) or a serum albumin or other polypeptide portion that provides desirable properties such as improved pharmacokinetics, improved solubility or improved stability. In some embodiments, an ActRII-Fc fusion comprises a relatively unstructured linker positioned between the Fc domain and the extracellular ActRII domain. This unstructured linker can correspond to the roughly 15 amino acid unstructured region at the C-terminal end of the extracellular domain of ActRII (the “tail”), or it can be an artificial sequence of 1, 2, 3, 4 or 5 amino acids or a length of between 5 and 15, 20, 30, 50 or more amino acids that are relatively free of secondary structure, or a mixture of both. A linker can be rich in glycine and proline residues and may, for example, contain a single sequence of threonine/serine and glycines or repeating sequences of threonine/serine and glycines (e.g., TG4 or SG4 singlets or repeats). A fusion protein can include a purification subsequence, such as an epitope tag, a FLAG tag, a polyhistidine sequence, and a GST fusion. A soluble ActRII polypeptide can include one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent.

It is understood that different elements of the fusion proteins can be arranged in any manner that is consistent with the desired functionality. For example, an ActRII polypeptide can be place C-terminal to heterologous domain, or alternatively, a heterologous domain can be placed C-terminal to an ActRII polypeptide. The ActRII polypeptide domain and the heterologous domain need not be adjacent in a fusion protein, and additional domains or amino acid sequences can be included C- and/or N-terminal to either domain or between the domains.

In some embodiments, ActRII polypeptide comprises an amino acid sequence selected from the group consisting of SEQ ID NO: 1-4, 6-13, and any combinations thereof.

In some embodiments, ActRII polypeptide consists essentially of an amino acid sequence selected from the group consisting of SEQ ID NO: 1-4, 6-13, and any combinations thereof.

In some embodiments, ActRII polypeptide consists of an amino acid sequence selected from the group consisting of SEQ ID NO: 1-4, 6-13, and any combinations thereof.

In some embodiments, ActRII polypeptide comprises an amino acid sequence of SEQ ID NO: 6 or SEQ ID NO: 7.

In some embodiments, ActRII polypeptide consists essentially of an amino acid sequence of SEQ ID NO: 6 or SEQ ID NO: 7.

In some embodiments, ActRII polypeptide consists of an amino acid sequence of SEQ ID NO: 6 or SEQ ID NO: 7.

ActRII polypeptides can be glycosylated, PEGylated, or linked to another nonproteinaceous polymer. For example, the ActRII polypeptides can be linked to one of a variety of nonproteinaceous polymers, e.g., polyethylene glycol, polypropylene glycol, or polyoxyalkylenes, in the manner set forth in U.S. Pat. No. 4,640,835; No. 4,496,689; No. 4,301,144; No. 4,670,417; No. 4,791,192; and No. 4,179,337, content of all of which is incorporated herein by reference in their entirety. The ActRII polypeptides can be chemically modified by covalent conjugation to a polymer to increase their circulating half-life. Exemplary polymers, and methods to attach them to peptides, are also shown in U.S. Pat. No. 4,766,106; No. 4,179,337; No. 4,495,285; and No. 4,609,546, content of all of which is incorporated herein by reference in their entirety.

The ActRII polypeptides can be modified to have an altered glycosylation pattern (i.e., altered from the original or native glycosylation pattern). As used herein, “altered” means having one or more carbohydrate moieties deleted, changed, and/or having one or more glycosylation sites added to the original sequence. Addition of glycosylation sites to polypeptides can be accomplished by altering the amino acid sequence to contain glycosylation site consensus sequences well known in the art. Another means of increasing the number of carbohydrate moieties is by chemical or enzymatic coupling of glycosides to the amino acid residues. These methods are described in Int. Pat. App. Pub. No. WO 87/05330, and in Aplin et al. (1981) Crit. Rev. Biochem. 22:259-306, content of both of which is incorporated herein by reference in their entirety. Removal of any carbohydrate moieties present on ActRII may be accomplished chemically or enzymatically as described by Hakimuddin et al. (1987) Arch. Biochem. Biophys. 259:52; Edge et al. (1981)Anal. Biochem. 118:131 and by Thotakura et al. (1987) Meth. Enzymol. 138:350, content of both of which is incorporated herein by reference in their entirety.

In some embodiments, the ActRII polypeptides comprise one or more modifications that are capable of stabilizing the polypeptides. For example, such modifications can enhance the in vitro half life, enhance circulatory half life, and/or reduce proteolytic degradation of polypeptides. Such stabilizing modifications include, but are not limited to, fusion proteins (including, for example, fusion proteins comprising an ActRII polypeptide and a stabilizer domain) and altered glycosylation patterns.

As used herein, the term “sequence similarity,” in all its grammatical forms, refers to the degree of identity or correspondence between nucleic acid or amino acid sequences that may or may not share a common evolutionary origin.

As used herein, the term “Fc domain” refers to the C-terminal fragment of an immunoglobulin generated by proteolysis with papain, or a functional equivalent derived therefrom. The terms “Fc domain,”“Fc portion,” and “Fc region” are used interchangeably herein. Generally, Fc domain comprises from about amino acid (aa) 230 to about aa 447 of γ chain or its counterpart sequence in other types of antibody heavy chains (e.g., α, δ, ε, and μ for human antibodies), or a naturally occurring allotype thereof. The terms “non-human Fc domain,” “non-human Fc portion,” and “non-human Fc region” refer to the corresponding C-terminal fragment of a non-human antibody heavy chain (e.g., from mouse, rat, goat, or rabbit).

The term “Fc domain” should be understood to encompass recombinantly produced Fc fragments, including those derived from any antibody isotype, e.g., IgG, IgA, IgE, IgM, and any of the isotype subclasses. In some embodiments, the Fc domain is a murine Fc domain, i.e., derived from murine immunoglobulins. In some other embodiments, the Fc domain is a human Fc domain. In still some other embodiments, the Fc domain is a humanized Fc domain. By humanized is meant a non-human Fc domain which has been mutated as not to be immunogenic or toxic in humans. Skilled artisan is well aware of methods for humanizing non-human Fc domains.

In some embodiments, the Fc domain comprises the amino acid sequence of SEQ ID NO: 5. In some embodiments, the amino acid sequence of SEQ ID NO: 5 has a mutation at residues position 43, 100, and/or 212 (these positions are underlined in SEQ ID NO: 5 in Table 1). In some embodiments, the mutant Fc domain comprises Ala at residues position 43, 100 and/or 212. In certain cases, the mutant Fc domain having one or more of these mutations (e.g., Asp43 mutation) has reduced ability of binding to the Fey receptor relative to a wildtype Fc domain. In other cases, the mutant Fc domain having one or more of these mutations (e.g., Asn212 mutaiton) has increased ability of binding to the MHC class I-related Fc-receptor (FcRN) relative to a wildtype Fc domain.

The skilled artisan is well aware that can use a peptide bond replacement to make a more stable peptide, e.g. ActRII polypeptide and/or Fc domains. Exemplary peptide bond replacements include, but are not limited to, urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, and olefinic group. Accordingly, in some embodiments, one or more of the peptide bonds in the ActRII polypeptide and/or fusion protein can be replaced with a peptide bond replacement. The peptide bond can also be replaced by a linker.

In some embodiments, the ActRII polypeptide comprises at least one (e.g., 1, 2, 3, 4, 5, 6, 7, 8, 9, 10 or more) amino acid selected from the group consisting of homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, and derivatives thereof.

In some embodiments, the ActRII polypeptide comprises at least one (e.g., 1, 2, 3, 4, 5, 6, 7, 8, 9, 10 or more) D-amino acid. D-amino acid can be present at any position in the peptide, for example reading from the N-terminal at position 1, 2, 3, 4, 5, 6, 7, 8, 9, 10, 11, 12, 13, 14, 15, 16, 17, 18, 19, 20, 21, 22, 23, 24, or 25. When more than one D-amino acid is present in a peptide, they can be positioned next to or not next to each other.

The ActRII polypeptide can also comprise one or more (e.g., 1, 2, 3, 4, 5, 6, 7, 8, 9, 10 or more) of beta-amino acids. Beta-amino acid can be present at any position in the peptide, for example reading from the N-terminal at position 1, 2, 3, 4, 5, 6, 7, 8, 9, 10, 11, 12, 13, 14, 15, 16, 17, 18, 19, 20, 21, 22, 23, 24, or 25. When more than one beta-amino acid is present in a peptide, they can be positioned next to each other or next to another amino acid.

The choice of including a modification into a ActRII polypeptide depends, in part, on the desired characteristics of the ActRII polypeptide. For example, the incorporation of one or more (D)-amino acids can confer increasing stability on the ActRII polypeptide in vitro or in vivo, thus affecting shelf-life, serum half-life or bioavailability. The incorporation of one or more (D)-amino acids also can increase or decrease the binding activity of the ActRII polypeptide as determined, for example, using the binding assays by methods well known in the art. In some cases it is desirable to design a ActRII polypeptide which retains activity for a longer period of time, for example, when designing a ActRII polypeptide to administer to a subject. In these cases, the incorporation of one or more (D)-amino acids or replacement of amide backbone linkages in the peptide can stabilize the peptide against endogenous peptidases in vivo, thereby prolonging the subject's exposure to the peptide.

As used herein, the term “amino acid equivalent” refers to compounds which depart from the structure of the naturally occurring amino acids, but which have substantially the structure of an amino acid, such that they can be substituted within a peptide which retains is activity, e.g., biological activity. Thus, for example, in some embodiments amino acid equivalents can also include amino acids having side chain modifications or substitutions, and also include related organic acids, amides or the like.

The ActRII polypeptide can be synthesized by using well known methods including recombinant methods and chemical synthesis of peptides and polypeptides. Recombinant methods of producing a peptide through the introduction of a vector including nucleic acid encoding the peptide into a suitable host cell is well known in the art, such as is described in Sambrook et al., Molecular Cloning: A Laboratory Manual, 2d Ed, Vols 1 to 8, Cold Spring Harbor, N.Y. (1989); M. W. Pennington and B. M. Dunn, Methods in Molecular Biology: Peptide Synthesis Protocols, Vol 35, Humana Press, Totawa, N.J. (1994), contents of both of which are herein incorporated by reference. Peptides can also be chemically synthesized using methods well known in the art. See for example, Merrifield et al., J. Am. Chem. Soc. 85:2149 (1964); Bodanszky, M., Principles of Peptide Synthesis, Springer-Verlag, New York, N.Y. (1984); Kimmerlin, T. and Seebach, D. J. Pept. Res. 65:229-260 (2005); Nilsson et al., Annu. Rev. Biophys. Biomol. Struct. (2005) 34:91-118; W. C. Chan and P. D. White (Eds.) Fmoc Solid Phase Peptide Synthesis: A Practical Approach, Oxford University Press, Cary, N.C. (2000); N. L. Benoiton, Chemistry of Peptide Synthesis, CRC Press, Boca Raton, Fla. (2005); J. Jones, Amino Acid and Peptide Synthesis, 2^{nd} Ed, Oxford University Press, Cary, N.C. (2002); and P. Lloyd-Williams, F. Albericio, and E. Giralt, Chemical Approaches to the synthesis of peptides and proteins, CRC Press, Boca Raton, Fla. (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

Newly synthesized peptides can be purified, for example, by high performance liquid chromatography (HPLC), and can be characterized using, for example, mass spectrometry or amino acid sequence analysis.

Synthesis of beta-amino acids and their derivatives are described for example in Basler B, Schuster O, and Bach T. Conformationally constrained beta-amino acid derivatives by intramolecular [2+2]-photocycloaddition of a tetronic acid amide and subsequent lactone ring opening. J. Org. Chem. (2005) 70(24):9798-808; and Murray J K, Farooqi B, Sadowsky J D, Scalf M, Freund W A, Smith L M, Chen J, Gellman S H. Efficient synthesis of a beta-peptide combinatorial library with microwave irradiation. JACS (2005) 127(38):13271-80.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, at least one symptom of a disease or disorder is alleviated by at least 5%, at least 10%, at least 20%, at least 30%, at least 40%, or at least 50%.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of congenital myopathy.

In addition, the methods described herein can be used to treat domesticated animals and/or pets. A subject can be male or female. A subject can be one who has been previously diagnosed with or identified as suffering from or having a disorder associated with a congenital myopathy, or one or more complications related to such disease but need not have already undergone treatment.

A subject can be one who is currently undergoing treatment with an ActRII inhibitor for a disease or disorder not related to a congenital myopathy. In such situations, a method described herein comprises modulating the dosage and/or the dosing schedule of ActRII inhibitor administration.

### Pharmaceutical Compositions

For administration to a subject, the ActII inhibitors can be formulated in pharmaceutically acceptable compositions. These pharmaceutically acceptable compositions comprise a therapeutically-effective amount of one or more of the inhibitors formulated together with one or more pharmaceutically acceptable carriers (additives) and/or diluents. The ActRIIB inhibitors can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

“PEG” means an ethylene glycol polymer that contains about 20 to about 2000000 linked monomers, typically about 50-1000 linked monomers, usually about 100-300. Polyethylene glycols include PEGs containing various numbers of linked monomers, e.g., PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof.

The inhibitors can be formulated in a gelatin capsule, in tablet form, dragee, syrup, suspension, topical cream, suppository, injectable solution, or kits for the preparation of syrups, suspension, topical cream, suppository or injectable solution just prior to use. Also, inhibitors can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting an ActRIIB inhibitor with one or more excipients or carriers. In general, the formulations are prepared by uniformly and intimately bringing into association one or more inhibitors of ActRIIB with liquid excipients or finely divided solid excipients or both, and then, if appropriate, shaping the product.

The preparative procedure may include the sterilization of the pharmaceutical preparations. The compounds may be mixed with auxiliary agents such as lubricants, preservatives, stabilizers, salts for influencing osmotic pressure, etc., which do not react deleteriously with the compounds.

Examples of injectable form include solutions, suspensions and emulsions. The compounds of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. A suitable carrier is 5% dextrose in saline. Frequently, it is desirable to include additives in the carrier such as buffers and preservatives or other substances to enhance isotonicity and chemical stability.

In some embodiments, ActRII inhibitors can be administrated encapsulated within liposomes. The manufacture of such liposomes and insertion of inhibitors into such liposomes are well known in the art.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like.

Examples of bases useful for the formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives.

The compositions can be given as a bolus dose, to maximize the circulating levels for the greatest length of time after the dose. Continuous infusion may also be used after the bolus dose.

For administration by inhalation, the inhibitors can be delivered in the form of an aerosol spray from pressured container or dispenser which contains a suitable propellant, e.g., a gas such as carbon dioxide, or nebulizer.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of active compound calculated to produce the desired therapeutic effect in association with the required pharmaceutical carrier.

As used herein, the term “administer” refers to the placement of a composition into a subject by a method or route which results in at least partial localization of the composition at a desired site such that desired effect is produced. An inhibitor can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

The inhibitor can be administrated to a subject in combination with a pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., N.Y.; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

The inhibitor and the pharmaceutically active agent can be administrated to the subject in the same pharmaceutical composition or in different pharmaceutical compositions (at the same time or at different times). When administrated at different times, the compound and the pharmaceutically active agent can be administered within 5 minutes, 10 minutes, 20 minutes, 60 minutes, 2 hours, 3 hours, 4, hours, 8 hours, 12 hours, 24 hours of administration of the other When the inhibitor and the pharmaceutically active agent are administered in different pharmaceutical compositions, routes of administration can be different.

The amount of inhibitor which can be combined with a carrier material to produce a single dosage form will generally be that amount of the compound which produces a therapeutic effect. Generally out of one hundred percent, this amount will range from about 1% to 99% of inhibitor, preferably from about 5% to about 70%, most preferably from 10% to about 30%.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. For example, an amount of a compound administered to a subject that is sufficient to produce a statistically significant, measurable change in at least one symptom of a congenital myopathy.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, GDF-8 binding assays, and immunological assays.

The dosage may be determined by a physician and adjusted, as necessary, to suit observed effects of the treatment. Generally, the compositions are administered so that inflammasome inhibitor is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μ/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg,8 mg/kg to 10 mg/kg, 9 mg/kg to 10 mg/kg , and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg , and the like.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the inhibitor. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Materials and Methods

Live Animal Studies: All studies were performed with approval from the institutional animal care and utilization committee at Children's Hospital Boston (Boston, Mass.). MTM1/HSA mice (Buj-Bello, et al., Hum Mol Genet. 2002, 11:2297-2307) were a gift from Anna Buj-Bello and colleagues at the Université de Strasbourg, College de France (Illkirch, France), and were subsequently back-crossed onto a C57BL6 background. Genotyping of these MTM1/C57BL6 mice (Mtm1δ4) was performed as previously described in Buj-Bello, et al., Hum Mol Genet. 2002, 11:2297-2307. Male wild-type and Mtm1δ4 mice were injected semiweekly beginning at 14 days of life with ActRIIB-mFC (also referred to as RAP-031; Acceleron Pharma Inc., Cambridge, Mass.) at a dose of 20 mg/kg. This dosage was chosen because it was above the level at which muscle hypertrophy plateaued in wild-type mice (data not shown). Vehicle-treated animals were injected with an equivalent volume of Tris-buffered saline solution. Treatment was continued until Mtm1δ4 animals were judged to be at end stage, and wild-type animals were sacrificed at equivalent time points. Behavior of wild-type and Mtm1δ4 animals was evaluated throughout the treatment period, and they were considered at end stage when they had either lost 20% of their highest body mass measurement or demonstrated complete inability to use their hindlimbs. The age at which Mtm1δ4 animals reached end stage was closely tracked to enable construction of Kaplan-Meier survival curves using commercially available software (Prism 4; GraphPad Software, Inc., San Diego, Calif.). Animals were weighed five times per week during the treatment period. For statistical analysis, running averages of the animals’ weight over 3 days were calculated to provide daily measurements of animal weight. Forelimb grip strength was measured weekly using a Chatillon grip force meter (Columbus Instruments International, Columbus, Ohio) by placing the animal on a horizontal grid and allowing it to pull away from the experimenter by using only its forelimbs. The average of three independent measurements, with a 1-minute recovery period between measurements, was used for subsequent statistical analysis. Antigravity hanging performance was tested three times weekly by placing the animals on a rigid mesh surface, inverting the surface at approximately 40 cm above their cage, and recording the amount of time necessary for the animal to fall back into the cage. Animals that did not fall within 60 seconds were lowered back into their cages. The maximum of three independent measurements, with a 1-minute recovery period between measurements, was used for subsequent statistical analysis. Footprint analysis was performed weekly by immersion of an animal's feet in nontoxic ink and allowing the animal to walk across an 8.5×11-inch piece of paper contained in an acrylic safety glass (Plexiglas) walkway. Footsteps and foot drags were counted, and the ratio of foot drags to foot steps was calculated for subsequent statistical analysis (Crawley, J. N., What's Wrong with My Mouse? (Behavioral Phenotyping of Transgenic and Knockout Mice). In: 2nd ed. Hoboken, N.J.: Wiley-Liss; 2007, p. 368).

Statistical Analysis: Statistical analysis was performed using commercially available software (Prism 4; GraphPad, Inc.). For statistical analysis of animal weight, forelimb grip strength, antigravity hanging performance, and foot drag, analysis of variance was performed, with Bonferroni posttests. For measurement of muscle weight and mean myofiber diameter, one-way analysis of variance was performed, with Bonferroni posttests. For survival data, statistical significance was evaluated using a log-rank test.

Western Blot: Tissues from the quadriceps, gastrocnemius, and triceps muscles and from elsewhere in the forelimbs and hindlimbs were frozen at necropsy and stored at −80° C. until analysis. Protein isolation and Western blot procedures were performed as previously described in Wattanasirichaigoon et al., Neurology. 2002, 59:613-617. Transferred proteins were probed with antibodies against myostatin (MAB788, 1:250 dilution; R&D Systems, Inc., Minneapolis, Minn.) and GAPDH [glyceraldehyde-3-phosphate dehydrogenase (6C5), 1:10,000 dilution; Abcam PLC, Cambridge, Mass.] and visualized using enhanced chemiluminescence. Adequacy of transfer was determined using Ponceau S staining. Quantification of protein levels normalized to GAPDH was performed using the program QuantityOne, version 4.2.1 (Bio-Rad Laboratories, Inc., Hercules, Calif.) on an Image Station 440 (Kodak DS; Eastman Kodak Co., Rochester, N.Y.).

Pathologic Evaluation and Tissue Collection: Animals were euthanized using CO₂ followed by cervical dislocation, per the regulations of the institutional animal care and utilization committee at Children's Hospital Boston. Animals were photographed externally and after removal of the skin from the torso and limbs. The quadriceps, gastrocnemius, triceps, soleus, extensor digitorum longus, tibialis anterior, and diaphragm muscles were removed and weighed. For subsequent ultrastructural studies, a small portion of the quadriceps muscle was fixed in 5% glutaraldehyde, 2.5% paraformaldehyde, and 0.06% picric acid in 0.2 mmol/L of cacodylate buffer, pH 7.4.

Histologic Evaluation: Eight-micrometer cross sections of isopentane-frozen quadriceps muscle were obtained midway down the length of the muscle and stained with H&E for evaluation using an Eclipse 50i microscope (Nikon Instruments Inc., Melville, N.Y.). Light microscopic images were captured using a SPOT Insight 4 Meg FW Color Mosaic camera and SPOT 4.5.9.1 software (Diagnostic Instruments Inc., Sterling Heights, Mich.).

For immunofluorescence studies, 8-μm frozen transverse sections of quadriceps muscle were double stained with rabbit antidystrophin antibodies (ab15277, 1:100; Abcam PLC) and mouse monoclonal antibodies against myosin heavy chain type 1 (Skeletal, Slow, clone NOQ7.5.4D, 1:100 dilution; Sigma Aldrich, St. Louis, Mo.) or types 2a (clone SC-71, 1:50 dilution) or 2b (clone BF-F3, 1:50 dilution; Developmental Studies Hybridoma Bank, University of Iowa, Iowa City). Secondary antibodies included fluorescein isothiocyanate-conjugated anti-mouse IgG or IgM (both 1:100; Sigma-Aldrich) and AlexaFluor-conjugated anti-rabbit IgG (1:50; Molecular Probes, Carlsbad, Calif.). Staining was evaluated using a Nikon Eclipse 90i microscope using NIS-Elements AR software (Nikon Instruments Inc.). For morphometric evaluation and estimation of fiber number, nonoverlapping fields of muscle immunostained for dystrophin were photographed using a Nikon Plan Fluor 4×/0.13 objective (Nikon Instruments Inc.). Fibers that stained positive were individually selected using NIS-Elements AR software, and fibers were automatically measured with respect to their minimum Feret (MinFeret) diameter. The MinFeret diameter is the smallest diameter across an ellipse, which corresponds to the “greatest distance between the opposite sides of the narrowest aspect of the fiber,” which is a measurement commonly used in morphometric analyses. See for example, Brooke, M. H. & Engel, W. K., Neurology. 1969; 19:591-605. An adequate number of fibers were counted to ensure measurements representative of the overall specimen, which involved a larger number of fibers counted in Mtm1δ4 animals (mean, 755; range, 238-1294 fibers) than in wild-type animals (mean, 340; range, 230-416 fibers) because of the small fiber size and the regional variability seen in the Mtm1δ4 animals. All MinFeret diameters for a given specimen were pooled for generation of frequency histograms and estimation of total number of fibers within the quadriceps muscle. In addition, the mean fiber MinFeret diameter for each specimen was calculated for subsequent statistical analysis.

For electron microscopy studies, tissue was fixed in 5% glutaraldehyde, 2.5% paraformaldehyde, 0.06% picric acid in 0.2M cacodylate buffer pH7.4. Fixed tissue was subjected to osmication, stained using uranyl acetate, dehydrated in alcohols, and embedded in TAAB Epon (Marivac Ltd., Halifax, Nova Scotia, Canada). Subsequently, 1-μm scout sections were stained with toluidine blue, and evaluated and photographed as described. Areas of interest were cut at 95-nm thickness using an ultracut microtome (Leica Camera AG, Solms, Germany), picked up on 100-m formvar-coated copper grids, stained with 0.2% lead citrate, and viewed and imaged using a Tecnai BioTwin Spirit Electron Microscope (FEI Co., Hillsboro, Oreg.).

### Results and Discussion

Myostatin Expression in Mtm1δ4 Mice: The relative levels of myostatin were investigated in untreated wild-type and Mtm1δ4 animals to ensure that the dosage would be adequate to inhibit the circulating myostatin in Mtm1δ4 mice. Similar amounts of myostatin protein were produced in Mtm1δ4 and wild-type mice at 43 days of life, although the levels observed in individual wild-type mice varied markedly (data not shown). The presence of similar levels of myostatin in wild-type and Mtm1δ4 mice suggests that ActRIIB-mFC can induce effective myofiber hypertrophy in Mtm1δ4 animals at dosages that are effective in wild-type mice. For administration of a soluble activin type IIB receptor to wild-type mice see, for example, S. M. Cadena et al. J Appl Physiol. 2010, 109:635-642 and B. M. Morrison et al., Exp Neurol. 2009, 217:258-268.

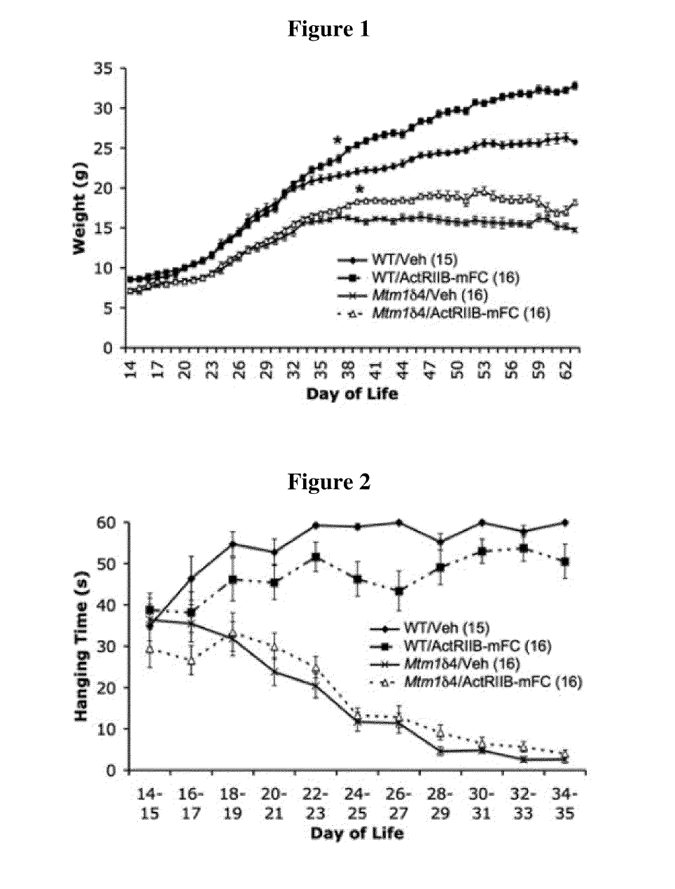

Weight Studies: In animals that received semiweekly injections of Tris-buffered saline solution (the ActRIIB-mFC vehicle), Mtm1δ4 animals were distinguishable from age-matched wild-type animals on the basis of weight at 20 days of life (P<0.05) (FIG. 1). These differences increased with age because of continued weight gain in wild-type animals in comparison with the plateau observed after 34 days of life in Mtm1δ4 animals. Compared with vehicle-treated mice, wild-type animals treated with semiweekly injections of ActRIIB-mFC, 20 mg/kg beginning at 14 days of life, showed significant weight gain after 36 days of life (P<0.05), and continued to gain weight with age. ActRIIB-mFC treated Mtm1δ4 animals initially exhibited a modest sustained weight gain, reaching a maximum of 124% at 53 days of life; however, the weight of these animals plateaued quickly and did not increase as observed in wild-type animals.

Antigravity Hanging Performance: At antigravity hanging assay, in which animals are suspended from a mesh grid until they either drop into the cage or have been hanging for 60 seconds, wild-type mice were able to hang for up to 60 seconds starting from 3 weeks of life (FIG. 2). Treatment of wild-type mice with ActRIIB-mFC led to a slight decrease in antigravity hanging performance, which was statistically significant only at 26 to 27 days of life (p<0.05). In contrast, vehicle-treated Mtm1δ4 animals exhibited impaired hanging performance at 2 weeks of life, which subsequently degenerated into nearly complete inability to remain suspended against gravity by 28 to 29 days of life. Treatment with ActRIIB-mFC did not measurably improve the antigravity hanging performance of Mtm1δ4 mice.

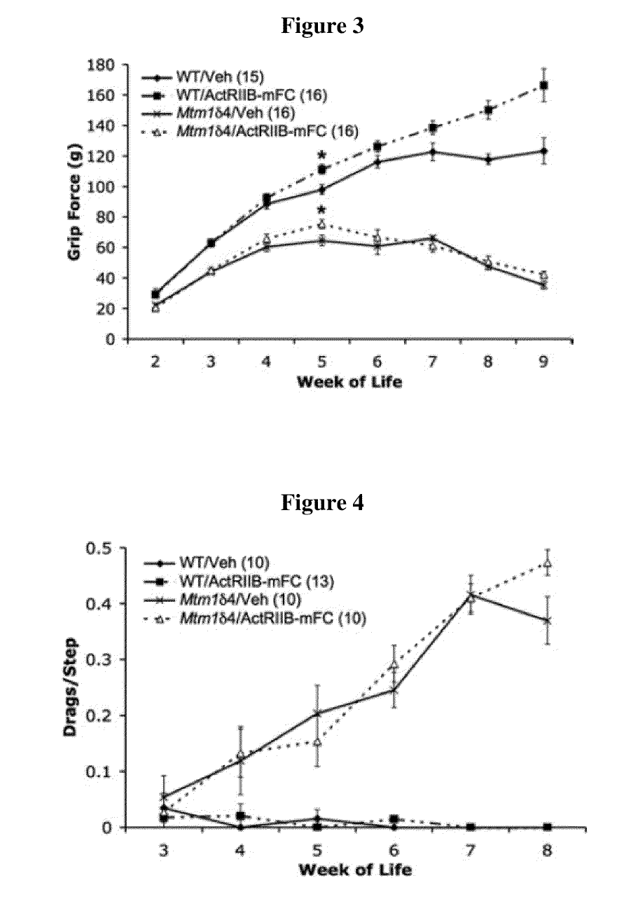

Forelimb Grip Strength: Mtm1δ4 animals exhibited consistently lower forelimb grip strength measurements at all ages tested (p<0.001) (FIG. 3). Forelimb grip force measurements in vehicle-treated wild-type animals showed consistent gains in grip strength as the animals aged, whereas the grip force of Mtm1δ4 animals was greatest at 5 weeks of life and then decreased as the disease progressed. Compared with their vehicle-treated counterparts, ActRIIB-mFC-treated wild-type animals also demonstrated increased grip strength as the treatment period progressed, with grip force up to 135% greater at 9 weeks of life, the latest time point tested. In ActRIIB-mFC-treated Mtm1δ4 animals, grip strength was transiently improved by 116% at 5 weeks of life (p<0.05), after which grip strength declined in both vehicle- and ActRIIB-mFC-treated Mtm1δ4 animals.

Footprint Analysis: Abnormalities of gait can be used to differentiate wild-type from Mtm1δ4 animals. Mtm1δ4 mice experience hindlimb weakness with disease progression, which can be visualized and quantified as foot drags on a footprinting assay. While foot drags are extremely uncommon in wild-type mice, the number of drags observed in Mtm1δ4 mice steadily increased after 3 weeks of life (p<0.05) (FIG. 4). Treatment with ActRIIB-mFC did not have any effect on the number of foot drags observed at footprint analysis.

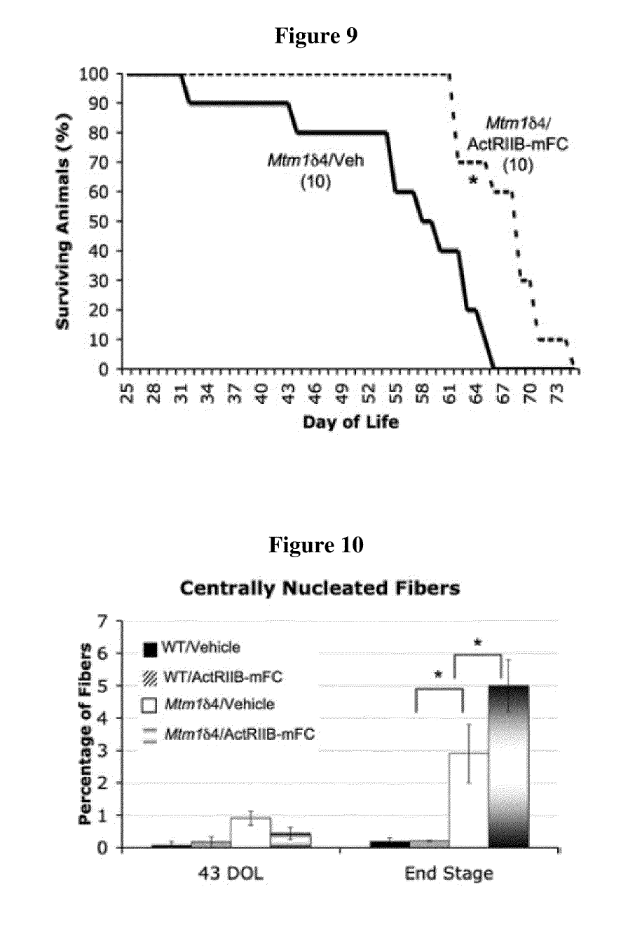

Survival: Similar to the first published reports using Mtm1δ4 mice on the HSA background, which stated that the survival of Mtm1δ4 animals ranged from 6 to 12 weeks (mean, 59 days), in the present study, untreated and vehicle-treated Mtm1δ4 animals demonstrated a maximum lifespan of approximately 8 to 9 weeks (mean, 56.1 days; range, 32-65 days). In comparisons of vehicle- and ActRIIB-mFC-treated Mtm1δ4 mice, treatment with ActRIIB-mFC significantly lengthened this lifespan by 17% (mean, 67.6 days; range, 61-74 days), with a shift in median survival from 58 days to 68 days (p<0.05) (FIG. 9). This survival benefit was due to both a decrease in the number of early deaths and delayed death in the oldest treated animals. This survival benefit was dose-dependent; reducing the dose to 5 mg/kg of ActRIIB-mFC in a pilot study of six animals resulted in an increase in median survival to 65 days (data not shown), which was still a significant improvement over that in the vehicle-treated animals (p<0.05).

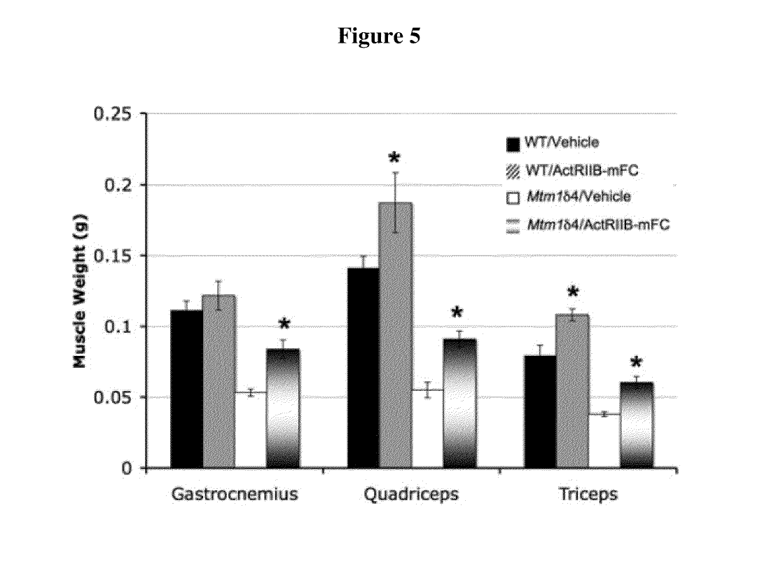

Gross Evaluation: Mtm1δ4 mice with late-stage disease can be easily differentiated from wild-type mice at gross and histologic examination. Mtm1δ4 mice are much smaller than age-matched wild-type mice and have proportionately smaller muscles (data not shown). At 43 days of life, when treated and untreated mice of both genetic backgrounds are easily distinguishable from their vehicle-treated counterparts on the basis of weight, ActRIIB-mFC treatment resulted in larger muscles in both wild-type and Mtm1δ4 mice (FIG. 5). ActRIIB-mFC treatment produced an increase in the weights of individual muscles in wild type mice, with a 128% increase in the weight of the quadriceps muscle at this time point. This increase in muscle weight occured to an even greater extent in the Mtm1δ4 mice at 43 days of life, with a 164% increase in the weight of the quadriceps muscle when comparing vehicle- and ActRIIB-mFC treated animals. Necropsy performed in treated animals at 8 to 10 weeks of life, which corresponded to the end-stage in treated and untreated Mtm1δ4 mice, revealed dramatic increases in muscle size in ActRIIB-mFC-treated wild-type animals. In contrast, vehicle- and ActRIIB-mFC-treated Mtm1δ4 mice were grossly indistinguishable from one another at end stage; both groups of mice were severely emaciated (data not shown). It should be noted, however, that the survival benefit produced by ActRIIB-mFC therapy prevented age-matching of these mice for this comparison. While the end-stage appearance of treated and untreated Mtm1δ4 mice was similar, the vehicle-treated mice were approximately 10 days younger than the ActRIIB-mFC-treated mice when the disease progressed to end stage. A clear difference (p<0.05) was seen between the muscle mass of gastrocnemius, quadriceps and triceps of vehicle and ActRIIB-mFC treated Mtm1δ4 mice (FIG. 5).

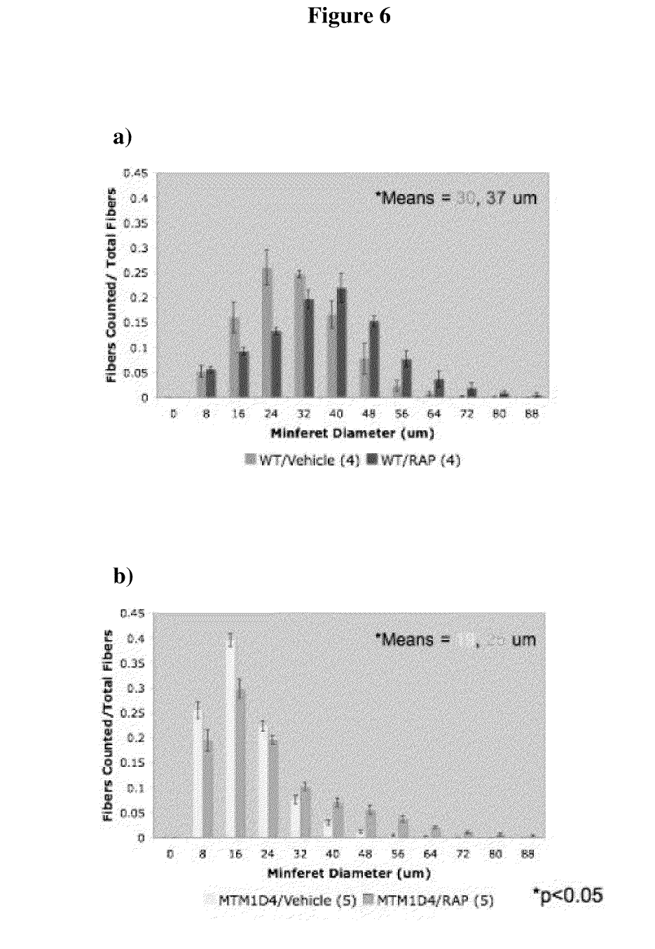

Histologic Examination: At histologic analysis, quadriceps muscle fibers of vehicle-treated Mtm1δ4 mice were dramatically smaller than those of age-matched wild-type mice, with mean fiber diameter of 19 and 30 mm, respectively, by 43 days of life (p<0.001). Although central nucleation is not a primary pathologic feature in this animal model, compared with muscles from wild-type mice, quadriceps muscles of Mtm1δ4 mice contained increased numbers of centrally nucleated fibers (p<0.05) (FIG. 10). The number of centrally nucleated fibers in Mtm1δ4 mice increased with age, irrespective of treatment. The greatest number of central nuclei was observed in ActRIIB-mFC-treated Mtm1δ4 mice, which may have been a consequence of their extended lifespan compared with that of vehicle-injected animals. Treatment of wild-type animals with ActRIIB-mFC produced an increase in the size of most fibers by 43 days of life, as evidenced by a 123% increase in mean fiber diameter and an increased percentage of large fibers in the quadriceps muscle (FIG. 6A).

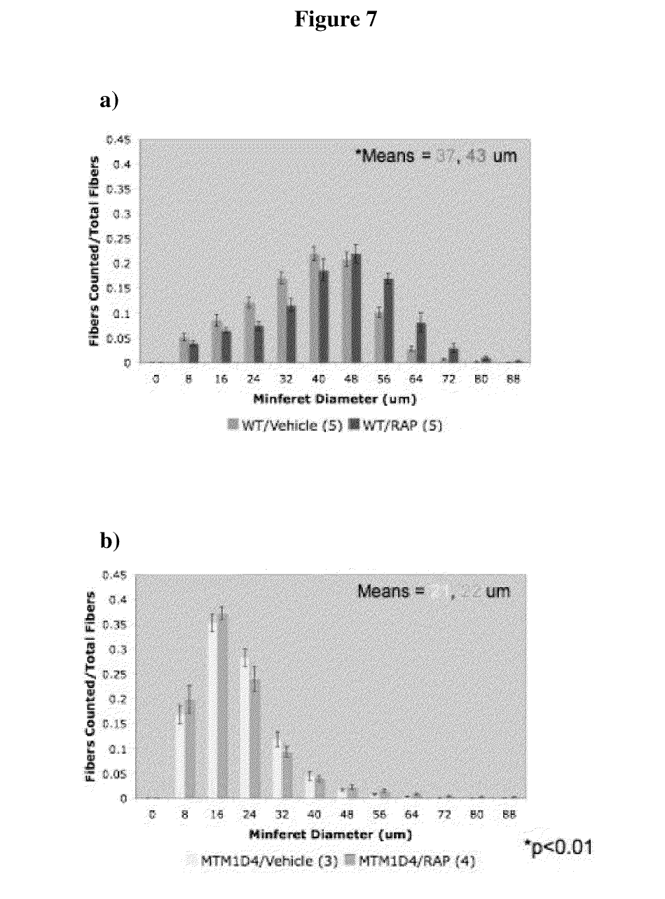

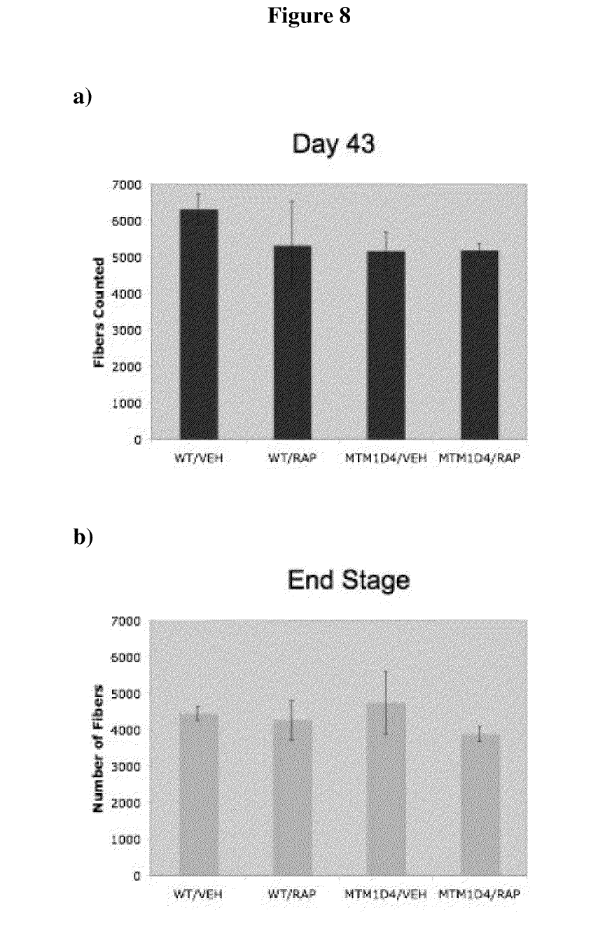

Similar differences were also seen in wild type mice that were sacrificed at later time points (FIG. 7). ActRIIB-mFC-treated Mtm1δ4 animals also showed marked hypertrophy of many muscle fibers when compared to their vehicle-treated counterparts, with a 132% increase in mean fiber diameter and a dramatic increase in the percentage of large muscle fibers (FIG. 6). In contrast, most of these large fibers were not seen in ActRIIB-mFC-treated mice at end stage, and vehicle- and ActRIIB-mFC-treated specimens were not easily distinguishable at this time point (data not shown). These results are consistent with a transient histologic improvement of Mtm1δ4 mice after ActRIIB-mFC treatment, which corresponds to the observed increases in animal and muscle weight at the same time point. In addition, estimations of myofiber number within the quadriceps muscle found no significant difference between WT and Mtm1δ4 mice, or between vehicle- and ActRIIB-mFC-treated mice of either genetic background. As shown in FIG. 8, the number of fibers was quite similar between all groups of mice.

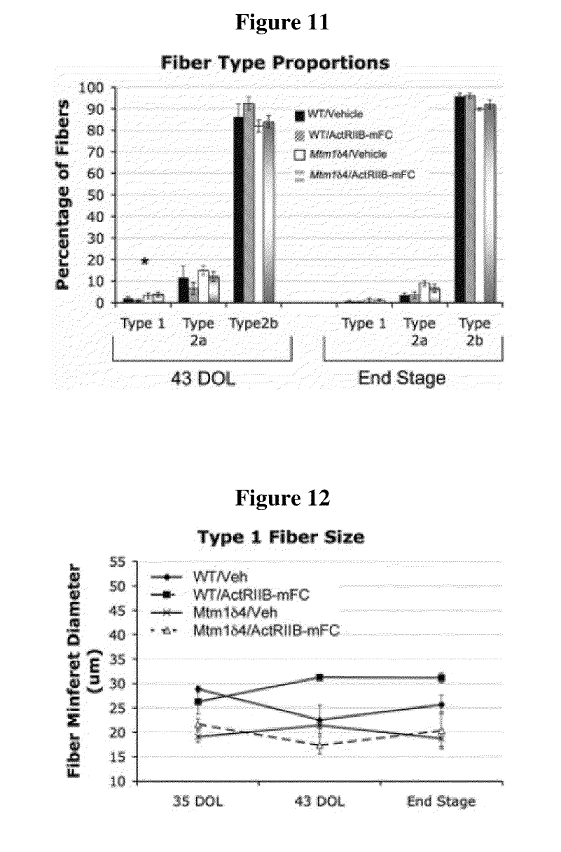

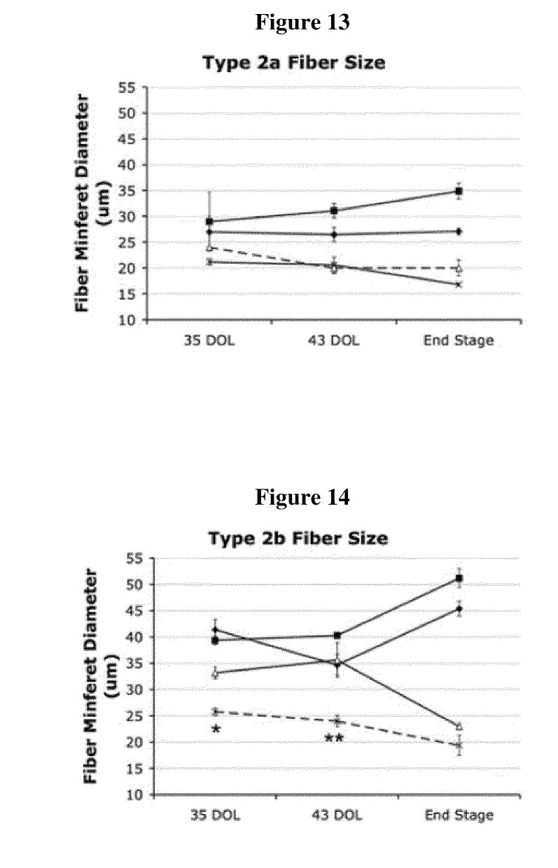

Immunostaining for oxidative (type 1 or 2a) or glycolytic (type 2b) myosin subtypes in the large fibers in the quadriceps muscles revealed that all types of muscle fibers experienced equivalent degrees of hypertrophy in response to ActRIIB-mFC treatment (FIGS. 11-14). Similar findings were also observed in wild-type mice that were sacrificed at end stage. ActRIIB-mFC-treated Mtm1δ4 animals also exhibited marked hypertrophy of a subpopulation of muscle fibers when compared with their vehicle-treated counterparts, with a 132% increase in mean fiber diameter overall. In contrast to the hypertrophy observed across all fiber types in wild-type animals, which was recently reported elsewhere (S. M. Cadena et al., J Appl Physiol. 2010, 109:635-642), hypertrophy in treated Mtm1δ4 mice was noted only in the glycolytic type 2b fibers, and there was a marked increase (149%) in mean type 2b fiber diameter in treated animals (p<0.001) at 35 and 43 days of life (FIGS. 11-14). Evaluation of animals with end-stage disease revealed small myofibers in both vehicle- and ActRIIB-mFC-treated animals, preventing histologic distinction between these two groups of mice at this time point. The percentage of type 1, 2a, and 2b fibers was similar between animals irrespective of genotype or treatment group (FIG. 11). These results are consistent with a transient histologic improvement in Mtm1δ4 mice after ActRIIB-mFC treatment, which corresponds to the observed increases in animal and muscle weight at the same stage. In addition, counts of myofibers within the quadriceps muscle revealed no significant difference between wild-type and Mtm1δ4 mice or between vehicle- and ActRIIB-mFC-treated mice of either genetic background. Diaphragm muscles collected from all treatment groups at 35 days of life were indistinguishable from one another, despite clear histologic differences between treatment groups in the quadriceps muscle at this time point (data not shown). These findings suggest that the diaphragm is not significantly affected in this disease model and that the cause of death in these animals may be related to dehydration or nutritional issues rather than to respiratory insufficiency.

Ultrastructural Examination: Ultrastructural tests of vehicle- and ActRIIB-mFC-treated mice of both genetic backgrounds were performed to evaluate the degree of structural organization in hypertrophied myofibers. At the ultrastructural level, the intracellular contents of vehicle- and ActRIIB-mFC-treated wild-type mice were indistinguishable from one another, with well-organized contractile filaments surrounded by pockets of mitochondria and stereotypically oriented triads consisting of T-tubules and sarcoplasmic reticulum (data not shown). Rare fibers from vehicle- and ActRIIB-mFC-treated Mtm1δ4 mice contained centrally located nuclei surrounded by aggregations of mitochondria and other disorganized organelles; however, these fibers were too uncommon to quantify accurately. Comparisons between vehicle- and ActRIIB-mFC-treated Mtm1δ4 mice revealed that some large fibers were present in ActRIIB-mFC-treated mice at 43 days of life; however, there was no difference in the organization of the contractile filaments between the two groups. The organization of the triads, which are composed of two elements of sarcoplasmic reticulum oriented perpendicular to a T-tubule (data not shown), was abnormal in the Mtm1δ4 mice because of loss of the appropriate orientation between these elements and an apparent increase in the number of stacks of sarcoplasmic reticulum between contractile elements. These abnormalities of the triads were unchanged by ActRIIB-mFC therapy. A subpopulation of degenerating atrophic myofibers with organellar and myofibrillar disorganization was observed in all Mtm1δ4 mice (data not shown). Some fibers of ActRIIB-mFC-treated Mtm1δ4 mice demonstrated ruffling and redundancy of the basement membrane consistent with atrophy of hypertrophic fibers (data not shown).

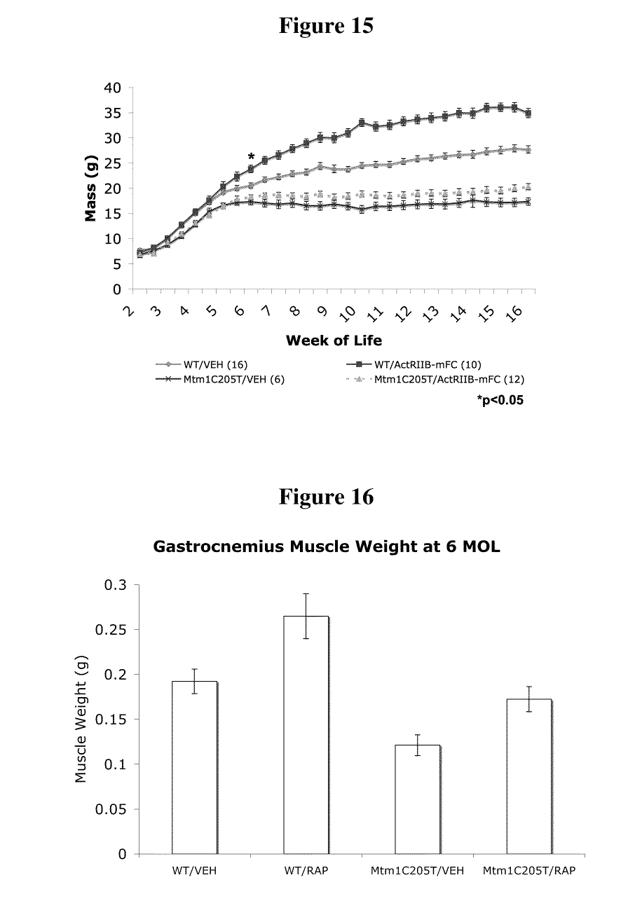

The inventors also studied treatment of a novel, less severe, murine model of myotubularin deficiency, the Mtm1C205T mouse, to show the potential of ActRIIB-mFC (RAP-031) therapy in cases of moderately severe myopathy. These mice incorporate a mutation that has been recurrently seen in XLMTM patients that survive past infancy, and the mice display less severe weakness and a longer lifespan than is seen in the Mtm1δ4 mice. On treatment of Mtm1C205T mice for up to 5.5 months, starting at 14 days of life and dosed twice per week at a dose of 20 mg/kg, the average mass of RAP-031 treated WT and Mtm1C205T mice was higher than their vehicle treated counterparts (FIG. 15). The animals were sacrificed at 6 months of age, and a significant increase in muscle mass was noted in the gastrocnemius muscle of RAP-031 treated Mtm1C205T mice (FIG. 16). Histologically, a subpopulation of fibers in the gastrocnemius muscle of RAP-031 treated Mtm1C205T mice showed marked hypertrophy (data not shown).

### Discussion

XLMTM is a currently untreatable severe congenital myopathy that is pathologically characterized by small myofibers and increased numbers of centrally nucleated myofibers. Based on the relationship between small fiber size and poor prognosis, the present study evaluated the therapeutic efficacy of ActRIIB-mFC, an agent that induces myofiber hypertrophy through inhibition of myostatin, in the Mtm1δ4 mouse. After treatment with ActRIIB-mFC, Mtm1δ4 mice exhibited increases in weight, muscle weight, and myofiber size, which behaviorally corresponded to a transient increase in forelimb grip strength and a 17% increase in lifespan.

Preclinical trials using myostatin loss or inhibition have been effective in ameliorating symptoms of weakness in some animal models of muscle injury, atrophy, and disease. Myostatin-deficient animals demonstrate less fibrosis and improved muscle regeneration after laceration injury (J. Zhu et al., J Biol Chem. 2007, 282:25852-25863) and experience less muscle atrophy in murine models of age-related sarcopenia (V. Siriett et al., Mol Ther. 2007, 15:1463-1470) and cancer-related cachexia (C. M. Liu et al., Gene Ther. 2008, 15:155-160 and M. E. Benny Klimek et al., Biochem Biophys Res Commun 391:1548-1554). Similarly, a recent study using ActRIIB-mFC in the SOD^{G93A} model of amyotrophic lateral sclerosis described muscle growth and delay in symptomatic onset in treated animals that was greater than that observed in SOD^{G93A}/Mstn^{-/-} mice (B. M. Morrison et al., Exp Neurol. 2009, 217:258-268). These studies indicate a role for myostatin inhibitors in conditions in which the primary disease process occurs outside of the myofiber. In addition, several myostatin inhibition strategies have improved muscle disease and/or function in the mdx mouse model of Duchenne muscular dystrophy (S. Bogdanovich et al., Nature. 2002, 420:418-421; K. T. Murphyet al., Am J Pathol 176:2425-2434; C. Qiao et al., Hum Gene Ther. 2009, 20:1-10; K. Tsuchida, Acta Myol. 2008, 27:14-18; and K. R. Wagner et al., Ann Neurol. 2002; 52:832-836) which suggests a therapeutic benefit in primary myopathies in which the basic defect involves myofiber structure and function. The only reported human trial using myostatin inhibition, which used antimyostatin antibodies in patients with adult muscular dystrophies (Becker muscular dystrophy, facioscapulohumeral dystrophy, and limb-girdle muscular dystrophy), did not demonstrate significant improvements in muscle strength or size (K. R. Wagneret al., Ann Neurol. 2008, 63:561-571). These results may reflect an impaired ability of adult patients with dystrophy to respond to myostatin inhibition, which was recently demonstrated in mdx mice (K. T. Murphy et al., Am J Pathol 176,2425-2434, or may simply reflect poor efficacy of that particular agent. Alternatively, additional ligands of ActRIIB may prevent myostatin inhibition alone from being therapeutically effective in human beings, which provides a strong rationale for pursuing studies of ActRIIB inhibition rather than myostatin inhibition in human beings.

In contrast to other conditions in which myostatin and ActRIIB inhibition has been tested, congenital myopathies represent a class of disease in which the primary disease process occurs within myofibers and in which the primary pathologic process causes weakness owing to the insufficient size and abnormal organization of elements within the myofiber. Thus, there is great potential for targeting of ActRIIB to be effective in patients with congenital myopathy, provided that the diseased muscle is capable of growth that causes functional improvement. The present data demonstrates the therapeutic benefits of myostatin inhibitors in patients with congenital myopathy, in particular because the Mtm1δ4 model of myotubular myopathy has a much more severe clinical course than that observed in most patients with congenital myopathy. While patients with XLMTM are usually born with significant weakness, the weakness is rarely progressive. In contrast, Mtm1δ4 mice are grossly normal at birth but undergo considerable symptomatic deterioration during the second month of life. In patients and animal models that do not undergo the catastrophic degeneration seen in Mtm1δ4 mice at approximately 2 months of life, greater or more prolonged benefits can be expected than what is describe herein in Mtm1δ4 mice.

These studies provide evidence that ActRIIB inhibition can extend lifespan in diseased animals, and represent the first description of a therapy that extends lifespan in Mtm1δ4 animals. Recent studies using ActRIIB-mFC in the SOD^{G93A} model of amyotrophic lateral sclerosis (B. M. Morrison et al., Exp Neurol. 2009, 217:258-268) and the hSMN2/delta7SMN/mSmn^{-/-} model of severe spinal muscular atrophy (C. J. Sumner et al., Hum Mol Genet. 2009; 18:3145-3152) described symptomatic improvements without extension of lifespan. It should be noted, however, that an extension of lifespan was observed after follistatin therapy in this mouse model, which, to inventor's knowledge, is the only other published report of lifespan extension while manipulating the myostatin pathway (F. F. Rose et al., Hum Mol Genet. 2009, 18:997-1005). The extension of lifespan observed in the present study was due to delay in the point at which animals experience weight loss and/or complete hindlimb paralysis. This prolongation of survival can correspond to a greater extension of lifespan in patients with XLMTM because of the availability of supportive care and the greater degree of symptomatic variability observed in these patients.

Recent studies have established that postnatal inhibition of the myostatin pathway produces myofiber hypertrophywithout the myocyte hyperplasia observed in myostatin knockout animals. See, for example, S. Bogdanovichet al., Nature. 2002, 420:418-421; L. Grobetet al., Mamm Genome. 1998, 9:210-213; L. A. Whittemore et al., Biochem Biophys Res Commun. 2003, 300:965-971; X. Zhu et al., FEBS Lett. 2000, 474:71-75; and A. C. McPherron, A. M. Lawler, & S. J. Lee, Nature. 1997, 387:83-90. While there is some evidence that myostatin inhibition during development can produce increased numbers of type 2b fibers in skeletal muscle (S. Girgenrath, K. Song & L. A. Whittemore, Muscle Nerve. 2005, 31:34-40), treatment of wild-type and Mtm1δ4 mice with ActRIIB-mFC did not change the number or percentage of type 1, 2a, and 2b fibers in the quadriceps muscles. These findings, which are consistent with a recent report of the effects of ActRIIB-mFC treatment in wild-type mouse muscle (S. M. Cadenaet al., J Appl Physiol. 2010, 109:635-642), support the theory that myocyte hyperplasia or changes in fiber type distribution do not occur with postnatal inhibition of myostatin or ActRIIB. In addition, while satellite cell behavior in response to treatment was not specifically examined, no evidence of satellite cell fusion or myoblast proliferation was observed at histologic evaluation of ActRIIB-mFC-treated muscles.

In contrast to the hypertrophy observed in all fiber types in treated wild-type animals (S. M. Cadenaet al., J Appl Physiol. 2010, 109:635-642), ActRIIB-mFC-treated Mtm1δ4 animals did not demonstrate hypertrophy in the oxidative type 1 and 2a myofibers at 43 days of life. The degree to which hypertrophy was observed in type 2b fibers, however, markedly exceeded the degree of hypertrophy observed in treated wild-type animals. These results indicate that myotubularin deficiency interferes with the hypertrophic response to myostatin inhibition in oxidative fibers. The growth pathways in type 1 and 2a myofibers are calcium-dependent, whereas hypertrophy in type 2b fibers occurs through the relatively calcium-independent Akt pathway. For a review see R. Bassel-Duby & E. N. Olson, Annu Rev Biochem. 2006, 75:19-37 and M. Sandri, Physiology (Bethesda) 2008, 23:160-170. Recent studies of myotubularin-deficient mice (L. Al-Qusairi et al., Proc Natl Acad Sci USA 2009, 106:18763-18768), fish (J. J. Dowlinget al., PLoS Genet. 2009, 5:e1000372), and dogs (A. H. Beggs et al., Proc Natl Acad Sci USA. 2010; 107:14697-14702) have described abnormalities in T-tubule and sarcoplasmic reticulum architecture and abnormalities in intracellular calcium handling (L. Al-Qusairi et al., Proc Natl Acad Sci USA 2009, 106:18763-18768). These abnormalities in intracellular calcium may interfere with activation of hypertrophic pathways in oxidative fibers and, thereby, prevent ActRIIB-mFC-induced hypertrophy in these fibers. The Akt pathway may not be affected by abnormalities of calcium homeostasis in myotubularin-deficient myofibers, which could explain the marked fiber type-specific hypertrophy observed in Mtm1δ4 animals. However, myotubularin seems to have a critical role in type 2b myofibers because these fibers eventually become atrophic between 43 days of life and disease end stage.

### REFERENCES

1. Heckmatt J Z, Sewry C A, Hodes D, Dubowitz V. Congenital centronuclear (myotubular) myopathy: a clinical, pathological and genetic study in eight children. Brain. 1985; 108(Pt 4):941-964.
2. Jungbluth H, Wallgren-Pettersson C, Laporte J. Centronuclear (myotubular) myopathy. Orphanet J Rare Dis. 2008; 3:26.
3. Buj-Bello A, Fougerousse F, Schwab Y, Messaddeq N, Spehner D, Pierson C R, et al. AAV-mediated intramuscular delivery of myotubularin corrects the myotubular myopathy phenotype in targeted murine muscle and suggests a function in plasma membrane homeostasis. Hum Mol Genet. 2008; 17:2132-2143.
4. Cao C, Backer J M, Laporte J, Bedrick E J, Wandinger-Ness A. Sequential actions of myotubularin lipid phosphatases regulate endosomal PI(3)P and growth factor receptor trafficking. Mol Biol Cell. 2008; 19:3334-3346.
5. Dowling J J, Vreede A P, Low S E, Gibbs E M, Kuwada J Y, Bonnemann C G, et al. Loss of myotubularin function results in T-tubule disorganization in zebrafish and human myotubular myopathy. PLoS Genet. 2009; 5:e1000372.
6. Laporte J, Hu L J, Kretz C, Mandel J L, Kioschis P, Coy J F, et al. A gene mutated in X-linked myotubular myopathy defines a new putative tyrosine phosphatase family conserved in yeast. Nat Genet. 1996; 13:175-182.
7. Pierson C R, Tomczak K, Agrawal P, Moghadaszadeh B, Beggs A H. X-linked myotubular and centronuclear myopathies. J Neuropathol Exp Neurol. 2005; 64:555-564.
8. Pierson C R, Agrawal P B, Blasko J, Beggs A H. Myofiber size correlates with MTM1 mutation type and outcome in X-linked myotubular myopathy. Neuromuscul Disord. 2007; 17:562-568.
9. Al-Qusairi L, Weiss N, Toussaint A, Berbey C, Messaddeq N, Kretz C, et al. T-tubule disorganization and defective excitation-contraction coupling in muscle fibers lacking myotubularin lipid phosphatase. Proc Natl Acad Sci USA. 2009; 106:18763-18768.
10. Buj-Bello A, Laugel V, Messaddeq N, Zahreddine H, Laporte J, Pellissier J F, et al. The lipid phosphatase myotubularin is essential for skeletal muscle maintenance but not for myogenesis in mice. Proc Natl Acad Sci USA. 2002; 99:15060-15065.
11. McPherron A C, Lawler A M, Lee S J. Regulation of skeletal muscle mass in mice by a new TGF-beta superfamily member. Nature. 1997; 387:83-90.
12. McCroskery S, Thomas M, Maxwell L, Sharma M, Kambadur R. Myostatin negatively regulates satellite cell activation and self-renewal. J Cell Biol. 2003; 162:1135-1147.
13. Joulia-Ekaza D, Cabello G. Myostatin regulation of muscle development: molecular basis, natural mutations, physiopathological aspects. Exp Cell Res. 2006; 312:2401-2414.
14. Clop A, Marcq F, Takeda H, Pirottin D, Tordoir X, Bibe B, et al. A mutation creating a potential illegitimate microRNA target site in the myostatin gene affects muscularity in sheep. Nat Genet. 2006; 38:813-818.
15. Lee S J, McPherron A C. Regulation of myostatin activity and muscle growth. Proc Natl Acad Sci USA. 2001; 98:9306-9311.
16. Lee S J, Reed L A, Davies M V, Girgenrath S, Goad M E, Tomkinson K N, et al. Regulation of muscle growth by multiple ligands signaling through activin type II receptors. Proc Natl Acad Sci USA. 2005; 102:18117-18122.
17. Cadena S M, Tomkinson K N, Monnell T E, Spaits M S, Kumar R, Underwood K W, et al. Administration of a soluble activin type IIB receptor promotes skeletal muscle growth independent of fiber type [published online ahead of print May 13, 2010]. J Appl Physiol. 2010; 109:635-642.
18. Morrison B M, Lachey J L, Warsing L C, Ting B L, Pullen A E, Underwood K W, et al. A soluble activin type IIB receptor improves function in a mouse model of amyotrophic lateral sclerosis. Exp Neurol. 2009; 217:258-268.
19. Sumner C J, Wee C D, Warsing L C, Choe D W, Ng A S, Lutz C, et al. Inhibition of myostatin does not ameliorate disease features of severe SMA mice. Hum Mol Genet. 2009; 18:3145-3152.
20. Buj-Bello A, Furling D, Tronchere H, Laporte J, Lerouge T, Butler-Browne G S, et al. Muscle-specific alternative splicing of myotubularin-related 1 gene is impaired in DM1 muscle cells. Hum Mol Genet. 2002; 11:2297-2307.
21. Crawley J N. What's Wrong with My Mouse? (Behavioral Phenotyping of Transgenic and Knockout Mice). In: 2nd ed. Hoboken, N.J.: Wiley-Liss; 2007; p. 368.
22. Wattanasirichaigoon D, Swoboda K J, Takada F, Tong H Q, Lip V, Iannaccone S T, et al. Mutations of the slow muscle alpha-tropomyosin gene, TPM3, are a rare cause of nemaline myopathy. Neurology. 2002; 59:613-617.
23. Brooke M H, Engel W K. The histographic analysis of human muscle biopsies with regard to fiber types. 4 (Children's biopsies). Neurology. 1969; 19:591-605.
24. Zhu J, Li Y, Shen W, Qiao C, Ambrosio F, Lavasani M, et al. Relationships between transforming growth factor-betal, myostatin, and decorin: implications for skeletal muscle fibrosis. J Biol Chem. 2007; 282:25852-25863.
25. Siriett V, Salerno M S, Berry C, Nicholas G, Bower R, Kambadur R, et al. Antagonism of myostatin enhances muscle regeneration during sarcopenia. Mol Ther. 2007; 15:1463-1470.
26. Liu C M, Yang Z, Liu C W, Wang R, Tien P, Dale R, et al. Myostatin antisense RNA-mediated muscle growth in normal and cancer cachexia mice. Gene Ther. 2008; 15:155-160.
27. Benny Klimek M E, Aydogdu T, Link M J, Pons M, Koniaris L G, Zimmers T A: Acute inhibition of myostatin-family proteins preserves skeletal muscle in mouse models of cancer cachexia. Biochem Biophys Res Commun 391:1548-1554.
28. Bogdanovich S, Krag T O, Barton E R, Morris L D, Whittemore L A, Ahima R S, et al. Functional improvement of dystrophic muscle by myostatin blockade. Nature. 2002; 420:418-421.
29. Murphy K T, Ryall J G M, Snell S M, Nair L, Koopman R, Krasney P A, Ibebunjo C, Holden K S, Loria P M, Salatto C T, Lynch G S: Antibody-directed myostatin inhibition improves diaphragm pathology in young but not adult dystrophic mdx mice. Am J Pathol 176:2425-2434.
30. Qiao C, Li J, Zheng H, Bogan J, Yuan Z, Zhang C, et al. Hydrodynamic limb vein injection of adeno-associated virus serotype 8 vector carrying canine myostatin propeptide gene into normal dogs enhances muscle growth. Hum Gene Ther. 2009; 20:1-10.
31. Tsuchida K. Myostatin inhibition by a follistatin-derived peptide ameliorates the pathophysiology of muscular dystrophy model mice. Acta Myol. 2008; 27:14-18.
32. Wagner K R, McPherron A C, Winik N, Lee S J. Loss of myostatin attenuates severity of muscular dystrophy in mdx mice. Ann Neurol. 2002; 52:832-836.
33. Wagner K R, Fleckenstein J L, Amato A A, Barohn R J, Bushby K, Escolar D M, et al. A phase I/II trial of MYO-029 in adult subjects with muscular dystrophy. Ann Neurol. 2008; 63:561-571.
34. Rose F F, Mattis V B, Rindt H, Lorson C L. Delivery of recombinant follistatin lessens disease severity in a mouse model of spinal muscular atrophy. Hum Mol Genet. 2009; 18:997-1005.
35. Grobet L, Poncelet D, Royo L J, Brouwers B, Pirottin D, Michaux C, et al. Molecular definition of an allelic series of mutations disrupting the myostatin function and causing double-muscling in cattle. Mamm Genome. 1998; 9:210-213.
36. Whittemore L A, Song K, Li X, Aghajanian J, Davies M, Girgenrath S, et al. Inhibition of myostatin in adult mice increases skeletal muscle mass and strength. Biochem Biophys Res Commun. 2003; 300:965-971.
37. Zhu X, Hadhazy M, Wehling M, Tidball J G, McNally E M. Dominant negative myostatin produces hypertrophy without hyperplasia in muscle. FEBS Lett. 2000; 474:71-75.
38. Girgenrath S, Song K, Whittemore L A. Loss of myostatin expression alters fiber-type distribution and expression of myosin heavy chain isoforms in slow- and fast-type skeletal muscle. Muscle Nerve. 2005; 31:34-40.
39. Bassel-Duby R, Olson E N. Signaling pathways in skeletal muscle remodeling. Annu Rev Biochem. 2006; 75:19-37.
40. Sandri M. Signaling in muscle atrophy and hypertrophy. Physiology (Bethesda). 2008; 23:160-170.
41. Beggs A H, Bohm J, Snead E, Kozlowski M, Maurer M, Minor K, et al. MTM1 Mutation associated with X-linked myotubular myopathy in Labrador retrievers. Proc Natl Acad Sci USA. 2010; 107:14697-14702.

All patents and other publications identified herein are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

We claim:

1. A method for treating a subject having a congenital myopathy, comprising administering to said subject an effective amount of an activin receptor type II (ActII) inhibitor.

2. The method of claim 1, wherein ActRII is ActRIIB.

3. The method of claim 1, wherein the inhibitor binds an ActRII ligand.

4. The method of claim 1, wherein the congenital myopathy is selected from the group consisting of myotubular myopathy, centronuclear myopathy, central core myopathy, nemaline myopathy, multiminicore myopathy, congenital fiber type disproportion, and any combinations thereof.

5. The method of claim 1, further comprising the step of selecting the subject before onset of administering the ActRII inhibitor, wherein the subject has been previously diagnosed with a congenital myopathy.

6. The method of claim 1, further comprising the step of diagnosing the subject for a congenital myopathy before onset of administering the ActRII inhibitor.

7. The method of claim 1, wherein the inhibitor is a ActRII polypeptide comprising the amino acid sequence selected from the group consisting of:
a. SEQ ID NOs: 1-4, 10, and 11;
b. a polypeptide comprising an amino acid sequence at least 75% identical to an amino acid sequence selected from SEQ ID NOs.: 1-4, 10, and 11; and
c. a polypeptide comprising at least 10 consecutive amino acids selected from SEQ ID NOs: 1-4, 10, and 11.

8. The method of claim 7, wherein the ActRII polypeptide is a soluble peptide.

9. The method of claim 7, wherein the ActRII polypeptide comprises at least modified amino acid and/or peptide bond.

10. The method of claim 7, wherein the ActRII polypeptide is a fusion protein comprising an ActRII polypeptide domain and one or more polypeptide portions that enhance one or more of in vivo stability, in vivo half life, uptake/administration, tissue localization or distribution, formation of protein complexes, and/or purification.

11. The method of claim 10, wherein the one or more polypeptide portions are fused to the carboxyl terminus of the ActRII polypeptide.

12. The method of claim 11, wherein the fusion protein includes a polypeptide portion selected from the group consisting of an immunoglobulin Fc domain and a serum albumin.

13. The method of claim 12, wherein the Fc domain has the sequence SEQ ID NO. 5.

14. The method of claim 13, wherein the fusion protein is selected from the group consisting of SEQ ID NO: 6, SEQ ID NO: 7, SEQ ID NO: 12, and SEQ ID NO: 13.

15. The method of claim 1, wherein the subject is a mammal.

16. The method of claim 15, wherein the subject is a human.

17. The method of claim 1, wherein the inhibitor is administrated by one or more of the methods selected from the group consisting of intravenously, intraperitoneally, subcutaneously, intramuscularly, orally, topically, by aerosol, and any combinations thereof.

18. The method of claim 1, wherein the inhibitor is administered in a range of about 1 μg/kg to about 150 mg/kg of body weight.

19. The method of claim 1, wherein the inhibitor is administered daily.

20. The method of claim 1, wherein the inhibitor is administrated with a pharmaceutically acceptable carrier.
